(19) United States
(12) Patent Application Publication
(10) Pub. No.: US 2013/0065936 A1
(43) Pub. Date: Mar. 14, 2013

(54) MODULATION OF BIOACTIVE EPOXY-FATTY ACID LEVELS BY PHOSPHODIESTERASE INHIBITORS

(71) Applicants: Bruce D. Hammock, Davis, CA (US); Ahmet Bora Inceoglu, Davis, CA (US); Karen Wagner, Davis, CA (US); Nils Helge Schebb, Davis, CA (US)

(72) Inventors: Bruce D. Hammock, Davis, CA (US); Ahmet Bora Inceoglu, Davis, CA (US); Karen Wagner, Davis, CA (US); Nils Helge Schebb, Davis, CA (US)

(73) Assignee: THE REGENTS OF THE UNIVERSITY OF CALIFORNIA, Oakland, CA (US)

(21) Appl. No.: 13/697,155

(22) PCT Filed: May 13, 2011
(86) PCT No.: PCT/US11/36511

§ 371 Date: Dec. 4, 2012

Related U.S. Application Data
(60) Provisional application No. 61/334,871, filed on May 14, 2010.
(60) Provisional application No. 61/347,777, filed on May 24, 2010.

(51) Int. Cl. C07D 207/267; A61K 31/4015

(52) U.S. Cl. CPC C07D 207/267
USPC 514/424

(57) ABSTRACT

The present invention provides method for increasing levels of epoxygenated fatty acids by administration of a phosphodiesterase inhibitor.

### CROSS-REFERENCE TO RELATED APPLICATIONS

The present application claims the benefit of U.S. Provisional Application No. 61/334,871, filed on May 14, 2010 and U.S. Provisional Application No. 61/347,777, filed on May 24, 2010, the disclosures of each of which are hereby incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with government support with National Institute on Environmental Health Sciences Grant R01 ES002710, and National Institute on Environmental Health Sciences Superfund Basic Research Program Grant P42 ES04699. The government has certain rights in the invention.

### FIELD OF THE INVENTION

The present invention provides methods for increasing the levels of epoxygenated fatty acids in an individual in need thereof by administering an inhibitor of phosphodiesterase.

### BACKGROUND OF THE INVENTION

Pain is a major health problem associated with numerous diseases, and it is by itself a pathological chronic condition. Although many analgesics are available, side effects and lack of wide spectrum efficacy justify the search for novel drugs. Remarkably, stabilization of epoxy-fatty acids (EFA) through inhibition of the soluble epoxide hydrolase (sEH) reduces pain. However, in the absence of an underlying painful state sEH inhibitors (sEHi) are inactive. Herein, it is describe how these molecules alter pain perception. A pain producing second messenger, cAMP surprisingly positively regulates the activity of sEHi. Concurrent inhibition of sEH and phosphodiesterases (PDE) dramatically increases pain threshold in non-inflamed rodents. Our findings demonstrate a novel mechanism of action of cAMP and sEHi in the pathophysiology of pain. The cross-talk between cAMP and EFA paves the way to new approaches to understand and control pain.

### BRIEF SUMMARY OF THE INVENTION

In one aspect, the present invention provides methods of increasing levels of epoxygenated fatty acids in a subject in need thereof comprising administering to the subject an inhibitor of a phosphodiesterase.

In a further aspect, the invention provides methods of obtaining analgesic, anti-convulsant, anti-depressant, anti-inflammatory, anti-hypertensive, cardioprotective, organ protective effects in a subject in need thereof, comprising administering to the subject an inhibitor of phosphodiesterase.

In a further aspect, the invention provides methods of reducing, inhibiting, delaying, mitigating, or preventing in a subject pain (e.g., inflammatory and/or neuropathic pain), seizures (e.g., epilepsy), depression, inflammation, hypertension, diabetes, diabetic neuropathy, hyperglycemia, cardiomyopathy, cardiac arrhythmia, cardiac hypertrophy, nephropathy, damage from stroke, chronic obstructive lung diseases (e.g., COPDs, asthma), niacin-induced flushing, eye disorders due to increased intraocular pressure (e.g., glaucoma) and vascular restenosis after angioplasty or stenosis of vascular stents by administering to the subject an inhibitor of phosphodiesterase.

With respect to the embodiments, in some embodiments, the ratio of epoxygenated fatty acids to dihydroxy fatty acids is increased without changing the levels of dihydroxy fatty acids.

In some embodiments, soluble epoxide hydrolase is not inhibited.

In some embodiments, the inhibitor of phosphodiesterase is a non-selective inhibitor of phosphodiesterase.

In some embodiments, the inhibitor of phosphodiesterase is an inhibitor of PDE4. For example, the inhibitor of PDE4 can be selected from the group consisting of rolipram, roflumilast, cilomilast, ariflo, HT0712, ibudilast, mesembrine, pentoxifylline, piclamilast, and combinations thereof.

In some embodiments, the inhibitor of phosphodiesterase is an inhibitor of PDE5.

In some embodiments, the inhibitor of phosphodiesterase is administered in a subtherapeutic dose. In some embodiments, the inhibitor of phosphodiesterase is administered in a therapeutically effective dose.

In some embodiments, an inhibitor of soluble epoxide hydrolase is co-administered. In some embodiments, the inhibitor of soluble epoxide hydrolase is administered in a subtherapeutic dose. In some embodiments, the inhibitor of soluble epoxide hydrolase is administered in a therapeutically effective dose. In some embodiments, the inhibitor of soluble epoxide hydrolase comprises a urea, carbamate or amide pharmacophore. In some embodiments, the inhibitor of soluble epoxide hydrolase has an IC₅₀ of less than 500 μM.

In some embodiments, the inhibitor of phosphodiesterase is administered in a subtherapeutic dose and the inhibitor of soluble epoxide hydrolase is administered in a subtherapeutic dose.

In some embodiments, the increased the epoxygenated fatty acids are cis-epoxyeicosantrienoic acids (“EETs”), epoxides of linoleic acid, epoxides of eicosapentaenoic acid (“EPA”) or epoxides of docosahexaenoic acid (“DHA”), or a mixture thereof.

In a related aspect, the invention provides methods for preventing, reducing or inhibiting undesirable side effects in a subject caused by administration of an inhibitor of phosphodiesterase (PDEi) while maintaining efficacy of the PDEi, comprising administration of a subtherapeutic amount of the PDEi in combination with an inhibitor of soluble epoxide hydrolase (sEHi). The sEHi can be administered in a therapeutically effective or subtherapeutic amount.

In a related aspect, the invention provides methods for preventing, reducing or inhibiting undesirable side effects in a subject caused by administration of an inhibitor of soluble epoxide hydrolase (sEHi) while maintaining efficacy of the sEHi, comprising administration of a subtherapeutic amount of the sEHi in combination with an inhibitor of phosphodiesterase (PDEi). The PDEi can be administered in a therapeutically effective or subtherapeutic amount.

Further embodiments of the methods are as described herein.

### DEFINITIONS

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases. As discussed further in a separate section below, while the use of unmodified EETs is the most preferred, derivatives of EETs, such as amides and esters (both natural and synthetic), EETs analogs, and EETs optical isomers can all be used in the methods of the invention, both in pure form and as mixtures of these forms. For convenience of reference, the term “EETs” as used herein refers to all of these forms unless otherwise required by context.

“Epoxide hydrolases” (“EH;” EC 3.3.2.3) are enzymes in the alpha beta hydrolase fold family that add water to 3-membered cyclic ethers termed epoxides. The addition of water to the epoxides results in the corresponding 1,2-diols (Hammock, B. D. et al., in Comprehensive Toxicology: Biotransformation (Elsevier, New York), pp. 283-305 (1997); Oesch, F. Xenobiotica 3:305-340 (1972)). Four principal EH's are known: leukotriene epoxide hydrolase, cholesterol epoxide hydrolase, microsomal EH (“mEH”), and soluble EH (“sEH,” EH2, previously called cytosolic EH). A mammalian gene, message, protein and activity for EH3 has been described and a gene for EH3. The leukotriene EH acts on leukotriene A4, whereas the cholesterol EH hydrates compounds related to the 5,6-epoxide of cholesterol. The microsomal epoxide hydrolase metabolizes monosubstituted, 1,1-disubstituted, cis-1,2-disubstituted epoxides and epoxides on cyclic systems to their corresponding diols. Because of its broad substrate specificity, this enzyme is thought to play a significant role in ameliorating epoxide toxicity. Reactions of detoxification typically decrease the hydrophobicity of a compound, resulting in a more polar and thereby excretable substance. EH3 appears to have very tissue limited distribution but does metabolize fatty acid epoxides.

“Soluble epoxide hydrolase” (“sEH”) is an epoxide hydrolase which in many cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). NCBI Entrez Nucleotide accession number L05779 sets forth the nucleic acid sequence encoding the protein, as well as the 5′ untranslated region and the 3′ untranslated region. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)). Soluble EH is only very distantly related to mEH and hydrates a wide range of epoxides not on cyclic systems. In contrast to the role played in the degradation of potential toxic epoxides by mEH, sEH is believed to play a role in the formation or degradation of endogenous chemical mediators. Unless otherwise specified, as used herein, the terms “soluble epoxide hydrolase” and “sEH” refer to human sEH.

Unless otherwise specified, as used herein, the terms “sEH inhibitor” (also abbreviated as “sEHi”) or “inhibitor of sEH” refer to an inhibitor of human sEH. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

The term “neuroactive steroid” or “neurosteroids” interchangeably refer to steroids that rapidly alter neuronal excitability through interaction with neurotransmitter-gated ion channels, and which may also exert effects on gene expression via intracellular steroid hormone receptors. Neurosteroids have a wide range of applications from sedation to treatment of epilepsy and traumatic brain injury. Neurosteroids can act as allosteric modulators of neurotransmitter receptors, such as GABA_{A}, NMDA, and sigma receptors. Progesterone (PROG) is also a neurosteroid which activates progesterone receptors expressed in peripheral and central glial cells. Several synthetic neurosteroids have been used as sedatives for the purpose of general anaesthesia for carrying out surgical procedures. Exemplary sedating neurosteroids include without limitation alphaxolone, alphadolone, hydroxydione and minaxolone.

By “physiological conditions” is meant an extracellular milieu having conditions (e.g., temperature, pH, and osmolarity) which allows for the sustenance or growth of a cell of interest.

“Micro-RNA” (“miRNA”) refers to small, noncoding RNAs of 18-25 nt in length that negatively regulate their complementary mRNAs at the posttranscriptional level in many eukaryotic organisms. See, e.g., Kurihara and Watanabe, Proc Natl Acad Sci USA 101(34):12753-12758 (2004). Micro-RNA's were first discovered in the roundworm C. elegans in the early 1990s and are now known in many species, including humans. As used herein, it refers to exogenously administered miRNA unless specifically noted or otherwise required by context.

The term “therapeutically effective amount” refers to that amount of the compound being administered sufficient to prevent or decrease the development of one or more of the symptoms of the disease, condition or disorder being treated.

The terms “prophylactically effective amount” and “amount that is effective to prevent” refer to that amount of drug that will prevent or reduce the risk of occurrence of the biological or medical event that is sought to be prevented. In many instances, the prophylactically effective amount is the same as the therapeutically effective amount.

“Subtherapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., to obtain analgesic, anti-convulsant, anti-depressant, anti-inflammatory, anti-hypertensive, cardioprotective, or organ protective effects), or that quantitatively is less than the established therapeutic dose for that particular pharmacological agent (e.g., as published in a reference consulted by a person of skill, for example, doses for a pharmacological agent published in the Physicians' Desk Reference, 62nd Ed., 2008, Thomson Healthcare or Brunton, et al., Goodman & Gilman's The Pharmacological Basis of Therapeutics, 11th edition, 2006, McGraw-Hill Professional). A “subtherapeutic dose” can be defined in relative terms (i.e., as a percentage amount (less than 100%) of the amount of pharmacologically active agent conventionally administered). For example, a subtherapeutic dose amount can be about 1% to about 75% of the amount of pharmacologically active agent conventionally administered. In some embodiments, a subtherapeutic dose can be about 75%, 50%, 30%, 25%, 20%, 10% or less, than the amount of pharmacologically active agent conventionally administered.

The term “co-administration” refers to the presence of both active agents in the blood at the same time. Active agents that are co-administered can be delivered concurrently (L e., at the same time) or sequentially.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

The terms “reduce,” “inhibit,” “relieve,” “alleviate” refer to the detectable decrease in symptoms of neuropathic pain, as determined by a trained clinical observer. A reduction in neuropathic pain can be measured by self-assessment (e.g., by reporting of the patient), by applying pain measurement assays well known in the art (e.g., tests for hyperalgesia and/or allodynia), and/or objectively (e.g., using functional magnetic resonance imaging or f-MRI). Determination of a reduction of neuropathic pain can be made by comparing patient status before and after treatment.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than the listed active agents, e.g., an inhibitor of sEHi and/or an EET and a PDEi.

### BRIEF DESCRIPTION OF THE DRAWINGS

FIGS. 1A-C illustrate that inhibitors of sEH block pain mediated by PGE₂. (A) Structurally different sEHIs—TPAU and TUPS—eliminate PGE₂-elicited pain (100 ng per paw in 10 μL) whereas NSAIDs or a steroidal drug do not (compare with FIG. 5). TPAU 10 mg/kg and TUPS 3 mg/kg were administered subcutaneously (s.c.) with PEG400 as vehicle in all panels (n>6 per group). Pain is measured by von Frey mechanical allodynia assay by a fully blinded experimenter and reported as percentage change from pre-PGE₂ baseline mechanical withdrawal threshold. Baseline mechanical withdrawal, responses were measured and sEHIs were administered s.c. 1 h before PGE₂. Administration of PGE₂ decreased withdrawal threshold by 60%. (B) Lack of effect of high doses of sEHI on saline solution injection into the paw (without PGE2) in rats. Acute pain responses measured by von Frey assay (baseline vs. time points ANOVA, P>0.1). All data are expressed as percentage of pre-PGE₂ baseline and presented as mean±SEM. (C) Elevation of fatty acid epoxide-to-diol ratio in rats by TPAU (10 mg/kg, n=6 per group; Table 3 shows quantity and identities of analytes and Table 5 shows structures of sEHIs). The dose of sEHI that greatly increased plasma EFAs (Table 3) failed to show any change in perceived pain in these animals.

FIGS. 2A-H illustrate that sEHIs act in a pain intensity-dependent manner. (A-C) Mean line graphs showing effect of a constant dose of TPAU (10 mg/kg s.c.) on different magnitudes of pain intensity (i.e., hyperalgesia) resulting from different doses of PGE₂. TPAU did not alter baseline mechanical withdrawal thresholds (compare with FIG. 1B). Doses of PGE₂ vary as indicated (10, 30, and 100 ng per paw). Following intraplantar administration of PGE₂, animals were immediately placed in acrylic chambers standing on a mesh screen. Mechanical withdrawal thresholds were measured 5, 10, 15, 30, 45, and 60 min after PGE₂ by a fully blinded experimenter. For the initial time points of 5 and 10 min after PGE₂ administration, one measurement per animal per time point was recorded because of the short time interval between the time points. For the rest of the time points, three measurements at 1-min intervals were recorded and averaged as the threshold (n≧6 in all groups). (D) Constant dose of sEHI is less efficacious when rats have less hyperalgesia but is more effective when hyperalgesia is severe (y axis, percent difference in mechanical withdrawal threshold from mean of corresponding PGE₂ group, measured by von Frey assay). (E-G) Mean line graphs showing effect of TUPS (3 mg/kg s.c.) on pain elicited by different doses of PGE₂. (n=6 in all groups) (H) A structurally different sEHI, TUPS, acts similarly to TPAU in reducing PGE₂-induced pain in an intensity dependent manner (y axis, percent difference in mechanical withdrawal threshold from mean of corresponding PGE₂ group measured by von Frey assay). PGE₂ naturally increases the levels of intracellular cAMP without the need to inhibit phosphodiesterases.

FIGS. 3A-B illustrate that elevation of cAMP by PDEi substitutes for a pain-generated factor and initiates sEHI-mediated increase in nociceptive thresholds. A synergistic reduction in acute pain perception produced by simultaneous administration of PDEi and sEHI is shown. In this experiment, because pain is not elicited through inflammation or neuropathy, the increase from baseline pain thresholds (i.e., hind paw withdrawal latency from a thermal stimulus) are reported. (A) Time course of acute effects by TPAU/rolipram combination in comparison with the lack of effect of TPAU itself. Rolipram is a PDE4-selective, CNS-permeable inhibitor (Krause, et al., (1988) Xenobiotica 18:561-571). (B) Dose dependence of PDE4i and sEHI/PDE4i treatment 1 h after administration (TPAU 10 mg/kg constant dose in all sEHI/PDEi groups). Here, pain-related behavior is measured by Hargreaves thermal withdrawal latency assay and reported as percent change from predrug baseline (FIG. 11 shows pain-related behavior measured by Randall-Selitto mechanical sensitivity assay). Notably, the combination treatment is more potent and efficacious than rolipram alone.

FIGS. 4 A-B illustrate that sEHI and PDEi act distinctly but both modulate the levels of bioactive lipids. (A) Bar graph of ratios of plasma epoxy to dihydroxy-FAs in PDE4i- and sEHI-treated rats. Sum of epoxy- and dihydroxy-metabolites from arachidonic acid (ARA), eicosapentaenoic acid, and docosahexaenoic acid (Tables 3 and 4) were used to calculate the ratio for each animal. Because C18:2 fatty acids were much higher in concentration, they were not included in this graph, but can be found in Table 3. Rolipram, TPAU, and coadministration of rolipram and TPAU significantly elevated the EFAs over dihydroxy-FAs (ANOVA followed by Dunnett two-sided t test, P<0.008). TPAU and pentoxifylline were administered at 10 mg/kg, all other compounds at 1 mg/kg. Samples were obtained 60 min after s.c. drug administration. (B) Different effects of sEHI and PDEi on lipid metabolites. Inhibition of sEH both increased the sum of total EFAs (all groups, ANOVA followed by Dunnett two-sided t test, P=0.001) and reduced the sum of total dihydroxy-FAs (P=0.04), whereas the PDEi largely increased the sum of total EFAs without impinging on the sum of total dihydroxy-FA levels (ANOVA vehicle vs. all PDEi, P>0.55). Tables 3 and 4 display detailed information on the identity and quantity of each analyte.

FIGS. 5A-B illustrate that a selective COX-2 inhibitor and a steroidal anti-inflammatory drug are ineffective in reducing pain produced by the COX product PGE₂. Pain was induced by a single intraplantar injection of PGE₂ (100 ng per paw in 10 μL solution containing 10% DMSO) into one hind paw of rat and quantified by von Frey assay. Drugs were administered following baseline measurements, 1 h before PGE₂ administration. COX inhibitors and steroidal anti-inflammatory drugs act by reducing COX enzyme activity and expression, respectively. Therefore, pain produced by a product downstream to cyclooxygenases is expectedly impervious to reversal by celecoxib (20 mg/kg s.c.) or dexamethasone (5 mg/kg s.c.). In contrast, TUPS and TPAU are effective in reducing pain in this system and work downstream from PGE₂ (FIG. 1). The same data are presented in two different ways. All data are presented as mean±SEM (n=6 in all groups). On some graphs, the SEM bars are not visible because they are smaller than the symbol representing the data point. (A) The y-axis is the difference in % change in mechanical withdrawal threshold compared to pre-PGE₂ administration. (B) The y-axis is the difference in percentage change in mechanical withdrawal threshold compared with before PGE₂ administration.

FIG. 6 illustrates plasma and brain tissue levels of sEHI. Both compounds were dissolved in PEG400 and administered s.c. A dose of 10 mg/kg of TPAU and a dose of 3 mg/kg TUPS were administered (n=4 per group).

FIGS. 7A-C illustrate pain dependency of sEHi mediated antihyperalgesia. Line graphs of PGE₂ elicited pain at three different doses and reversal by TPAU (10 mg/kg, in all panels) are shown. Pain was measured by von Frey assay. TPAU was administered subcutaneously immediately following baseline measurements at a single dose of 10 mg/kg 1 h prior to PGE₂. PGE₂ doses vary as indicated on the panels (10, 30 and 100 ng/paw). Following intraplantar PGE₂ administration animals were immediately placed in acrylic chambers standing on a mesh screen. Mechanical withdrawal thresholds were measured at the times indicated on the x-axis. For the initial time points of 5 and 10 min post PGE₂ one measurement per animal per time point was recorded because of the short time interval between the time points. For the rest of the time points three measurements at 1 min intervals were recorded and averaged as the threshold. These values were then converted to % response taking baseline measurements as 100%. The efficacy of TPAU increased as the amount of administered PGE₂ increased (n=6 in all groups).

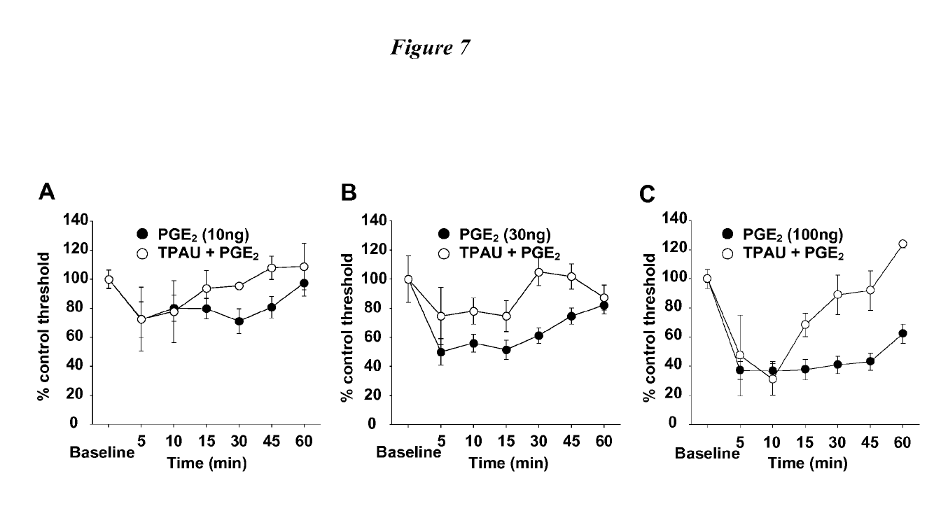

FIGS. 8A-C illustrate pain dependency of sEHi mediated antihyperalgesia. Line graphs of PGE₂ elicited pain at three different doses and reversal by TUPS (3 mg/kg, in all panels) are shown. Pain was measured by von Frey assay. TUPS was administered subcutaneously immediately following baseline measurements at a single dose of 3 mg/kg 1 h prior to PGE₂. PGE₂ doses vary as indicated on the panels (10, 30 and 100 ng/paw).

FIG. 9 illustrates the motor depressant effect of rolipram. Rolipram, as expected, led to a significant and dose-dependent decrease in voluntary movement in an open-field chamber even 1 h following administration. In contrast, TPAU treatment was indistinguishable from baseline activity. The combination of TPAU plus rolipram led to a similar degree of motor depression, but this depression as rolipram alone, unlike the synergistic analgesia produced, was not potentiated by the combination.

FIGS. 10A-D illustrate qualitative and quantitative differences between sEHI- and PDEi-mediated changes in EFAs and dihydroxy-fatty acids. Plasma levels of four groups of EFAs and their corresponding hydrolysis products as indicated on the panels are demonstrated in (A) 9,10-EpOME and 9,10-DiHOME and (B) 12,13-EpOME and 12,13-DiHOME. In particular, rolipram administration mediated no increase in leukotoxin (12,13-EpOME) levels but a significant elevation of the threefold more toxic leukotoxin diol (12,13-DiHOME) levels. Most other EFAs were elevated by rolipram, indicating that PDEis selectively modulate the levels of these bioactive lipid metabolites. The inhibitor of sEH elevated all EFAs quantified. These data are consistent with changes in EETs as demonstrated (C and D) (See, FIG. 4). Co-administration of rolipram and sEHI elevated the levels of EFAs. The undesirable increase in leukotoxin diol was ameliorated when rolipram and TPAU were co-administered.

FIGS. 11A-E illustrate rolipram- and sEHlVrolipram-mediated changes in nociceptive thresholds are pharmacologically distinct. (A) A selective COX-2 inhibitor, celecoxib, did not demonstrate any interaction with the PDE4 inhibitor rolipram, indicating that elevated cAMP is not required for COX inhibitors. (B) The GABA antagonist picrotoxin effectively antagonized the increases in thermal withdrawal latency produced by rolipram/AUDA but partially blocked rolipram. (C) The GABA antagonist picrotoxin effectively antagonized the increases in mechanical withdrawal threshold produced by rolipram/AUDA but not that of rolipram itself. Here, nociceptive thresholds are measured by Randall-Selitto mechanical sensitivity assay. (D) Line graph of competitive antagonism of the PDE41 rolipram produced antinociception by finasteride, a neurosteroid synthesis inhibitor. Here nociceptive thresholds are measured by Hargreaves thermal withdrawal latency assay. (E) Noncompetitive antagonism of rolipram by fluconazole (40 mg/kg), an inhibitor of epoxygenases in the CNS, and lack of antagonism by micanozole (40 mg/kg), a CNS-impermeable epoxygenase inhibitor. Nociceptive thresholds are measured by Hargreaves thermal withdrawal latency assay (n=6 per group in all panels).

### DETAILED DESCRIPTION

### 1. Introduction

Phosphodiesterase inhibitors (PDEi) are well known anti-inflammatory agents. Because these compounds lead to elevation of intracellular cAMP levels, the biological outcomes of inhibiting PDE are diverse. The present invention is based, in part, on the discovery that many different classes of isozyme selective PDEi lead to remarkable increases in the plasma levels of a broad range of epoxy-fatty acids (EFA). The magnitude of this increase is so dramatic that PDEi can elevate epoxy-fatty acids as well as highly potent inhibitors of soluble epoxide hydrolase. Data provided herein demonstrate the ability of PDEi to increase levels of EFA.

The soluble epoxide hydrolase (sEH) is the major enzyme that degrades the epoxygenated fatty acids to inactive molecules. Inhibitors of sEH stabilize the epoxy-fatty acids. The levels of epoxy-fatty acids are under tight control by multiple mechanisms, the sEH being one of these control mechanisms. Inhibition of sEH in many cases leads to a small increase in epoxygenated fatty acids levels but a large decrease in the degradation products of these bioactive lipids, the dihydroxyeicosanoids. Overall, the ratio of epoxy to dihydroxy fatty acids is a measurable parameter of therapeutic outcome in a number of disease models, including inflammatory disorders, pain and hypertension. Stabilization of natural epoxy-fatty acids (EFAs) through inhibition of the soluble epoxide hydrolase (sEH) reduces pain. However, in the absence of an underlying painful state, inhibition of sEH is ineffective. Surprisingly, a pain-mediating second messenger, cAMP, interacts with natural EFAs and regulates the analgesic activity of sEH inhibitors.

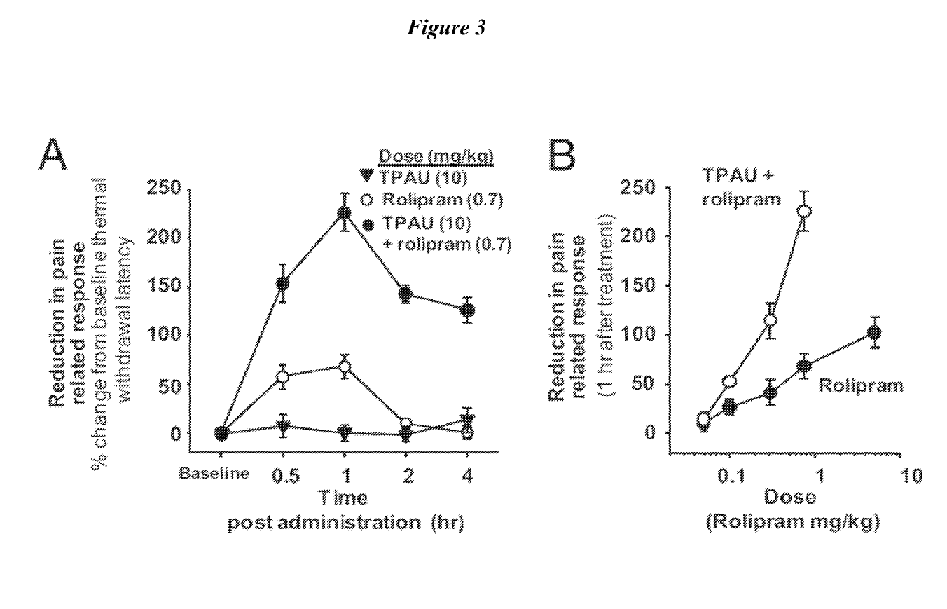

Concurrent inhibition of sEH and phosphodiesterase (PDE) dramatically reduced acute pain in a rodent model. The findings presented herein demonstrate a mechanism of action of cAMP and EFAs in the pathophysiology of pain. Furthermore, inhibition of various PDE isozymes, including PDE4, lead to significant increases in EFA levels through a mechanism independent of sEH, showing that the efficacy of commercial PDE inhibitors results in part from increasing EFAs. PDEi-induced increases in levels of EFA in a subject are further enhanced by the cooperative effects of concurrently administering an inhibitor of phosphodiesterase (PDEi) with an inhibitor of soluble epoxide hydrolase (sEHi). For example, FIG. 3 shows that the rapidly vanishing effect of PDEi is enhanced significantly by the sEHi. Once PDEi-mediated release of epoxy fatty acids occurred, the EFA levels were stabilized with sEHi and therefore remained active much longer.

The present invention shows for the first time that epoxy-fatty acid levels can be increased by inhibiting phosphodiesterases, independently of inhibiting sEH. Unlike the profile of potent sEH inhibitors, PDEi selectively elevate the epoxy-fatty acids but not influence the levels of dihydroxy fatty acids. The overall outcome of PDE inhibition is very similar to inhibition of sEH, a significant increase in epoxy to dihydroxy fatty acid ratio. Thus PDEi can be used to achieve the similar biological/pharmaceutical effects of sEHi. Furthermore, combining a sEHi with a PDEi further increases the epoxy to dihydroxy fatty acid ratio. This opens the route for new pharmacological method to target epoxy to dihydroxy fatty acid ratio as well as absolute concentrations of these very potent chemical mediators. Combining sEH inhibitors and PDE inhibitors, with one or both of the agents administered at a subtherapeutic dose can achieve therapeutic efficacy (e.g., in obtaining analgesic, anti-convulsant, anti-depressant, anti-inflammatory, anti-hypertensive, cardioprotective, organ protective effects) with reduced or eliminated side effects.

### 2. Methods of Increasing Epoxygenated Fatty Acids by Administering a PDEi

a. Conditions Subject to Treatment

Patients with conditions that will benefit by increasing their levels of epoxygenated fatty acids (e.g., in a biological sample, e.g., blood, plasma, serum and/or tissues) can by treated according to the present methods. Levels of epoxygenated fatty acids are increased by administration of an inhibitor of phosphodiesterase, alone or in combination with an inhibitor of soluble epoxide hydrolase.

Exemplary conditions subject to treatment (e.g., improvement, amelioration, delay or reversal of progression, reduction or inhibition of disease symptoms or severity) or prevention include conditions that can be treated, mitigated or prevented by administration of an inhibitor of soluble epoxide hydrolase, e.g., pain (e.g., inflammatory and/or neuropathic pain), seizures (e.g., epilepsy), depression, inflammation, hypertension, diabetes, diabetic neuropathy, hyperglycemia, cardiomyopathy, cardiac arrhythmia, cardiac hypertrophy, nephropathy, damage from stroke, chronic obstructive lung diseases (e.g., COPDs, asthma), niacin-induced flushing, eye disorders due to increased intraocular pressure (e.g., glaucoma) and vascular restenosis after angioplasty or stenosis of vascular stents. See, e.g., U.S. Patent Publication Nos. 2010/0074852, 2009/0216318, 2009/0018092, 2008/0279912, 2008/0249055, 2007/0117782, 2006/0148744, 2005/0282767, 2005/0222252 and 2003/0139469; and PCT Publication Nos. WO 2010/117951, WO 2010/030851, WO 2009/062073, WO 2008/101030, WO 2008/073130, WO 2007/022509, WO 2007/009001, WO 2006/133257, WO 2006/086108, WO 2005/094373, and WO 2005/089380, the disclosures of which are hereby incorporated herein by reference in their entirety for all purposes.

Because an inhibitor Of phosphodiesterase can be as effective as an inhibitor of soluble epoxide hydrolase in elevating levels of epoxygenated fatty acids, conditions that can be treated or prevented by administration of an inhibitor of soluble epoxide hydrolase can oftentimes be treated or prevented by administration of an inhibitor of phosphodiesterase (e.g., instead of administration of an inhibitor of soluble epoxide hydrolase).

Furthermore, for conditions treatable by either an inhibitor of phosphodiesterase (PDEi) or an inhibitor of soluble epoxide hydrolase (sEHi), administration of a combination of a PDEi and a sEHi can reduce undesirable side effects of either the PDEi or the sEHi while maintaining efficacy by allowing for administration of a subtherapeutic dose of one or both the PDEi and sEHi.

b. Phosphodiesterase Inhibitors (PDEi)

According to the present methods, levels of epoxygenated fatty acids (e.g., in blood, plasma, serum) are increased by administration of a phosphodiesterase inhibitor (PDEi).

The PDEi may or may not be selective, specific or preferential for cAMP. Exemplary PDEs that degrade cAMP include without limitation PDE3, PDE4, PDE7, PDE8 and PDE10. Exemplary cAMP selective hydrolases include PDE4, 7 and 8. Exemplary PDEs that hydrolyse both cAMP and cGMP include PDE1, 2, 3, 10 and 11. Isoenzymes and isoforms of PDEs are well known in the art. See, e.g., Boswell-Smith et al., Brit. J. Pharmacol. 147:S252-257 (2006), and Reneerkens, et al., Psychopharmacology (2009) 202:419-443, the contents of which are incorporated herein by reference.

In some embodiments, the PDE inhibitor is a non-selective inhibitor of PDE. Exemplary non-selective PDE inhibitors that find use include without limitation caffeine, theophylline, isobutylmethylxanthine, aminophylline, pentoxifylline, vasoactive intestinal peptide (VIP), secretin, adrenocorticotropic hormone, pilocarpine, alpha-melanocyte stimulating hormone (MSH), beta-MSH, gamma-MSH, the ionophore A23187, prostaglandin E1.

In some embodiments, the PDE inhibitor used specifically or preferentially inhibits PDE4. Exemplary inhibitors that selectively inhibit PDE4 include without limitation rolipram, roflumilast, cilomilast, ariflo, HT0712, ibudilast and mesembrine.

In some embodiments, the PDE inhibitor used specifically or preferentially inhibits a cAMP PDE, e.g., PDE4, PDE7 or PDE8. In some embodiments, the PDE inhibitor used inhibits a cAMP PDE, e.g., PDE1, PDE2, PDE3, PDE4, PDE7, PDE8, PDE10 or PDE11. Exemplary agents that inhibit a cAMP phosphodiesterase include without limitation rolipram, roflumilast, cilomilast, ariflo, HT0712, ibudilast, mesembrine, cilostamide, enoxamone, milrinone, siguazodan and BRL-50481.

In some embodiments, the PDE inhibitor used specifically inhibits PDE5. Exemplary inhibitors that selectively inhibit PDE5 include without limitation sildenafil, zaprinast, tadalafil, udenafil, avanafil and vardenafil.

Other means of inhibiting phosphodiesterase activity or gene expression can also be used in the methods of the invention. For example, a nucleic acid molecule complementary to at least a portion of a human phosphodiesterase gene (e.g., PDE3, PDE4, PDE7, PDE8 and PDE 10) can be used to inhibit phosphodiesterase gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440—R442 (1999); Baulcombe. D. Curr Biol 9:R599—R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2a, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of a phosphodiesterase enzyme, siRNAs to the gene encoding the phosphodiesterase can be specifically designed using computer programs. Exemplary nucleotide sequences encoding the amino acid sequences of the various phosphodiesterase isoforms are known and published, e.g., in GenBank, e.g., PDE1A (NM_{—}001003683.1→NP_{—}001003683.1 (isoform 2) and NM_{—}005019.3→NP_{—}005010.2 (isoform 1)); PDE1B (NM_{—}000924.3→NP_{—}000915.1 (isoform 1) and NM_{—}001165975.1→NP_{—}001159447.1 (isoform 2)); PDE2A (NM_{—}002599.3→NP_{—}002590.1 (isoform 1); NM_{—}001143839.2→NP_{—}001137311.1 (isoform 2) and NM_{—}001146209.1→NP_{—}001139681.1 (isoform 3)); PDE3A (NM_{—}000921.3→NP_{—}000912.3); PDE3B (NM_{—}000922.3→NP_{—}000913.2); PDE4A (NM_{—}001111307.1→NP_{—}001104777.1 (isoform 1); NM_{—}001111308.1→NP_{—}001104778.1 (isoform 2); NM_{—}001111309.1→NP_{—}001104779.1 (isoform 3); NM_{—}006202.2→NP_{—}006193.1 (isoform 4)); PDE4B (NM_{—}002600.3→NP_{—}002591.2 (isoform 1); NM_{—}001037341.1→NP_{—}001032418.1 (isoform 1); NM_{—}001037339.1—→NP_{—}001032416.1 (isoform 2); NM_{—}001037340.1→NP_{—}001032417.1 (isoform 3)); PDE4C-1 (NM_{—}000923.3→NP_{—}000914.2); PDE4C-2 (NM_{—}001098819.1→NP_{—}001092289.1); PDE4C-3 (NM_{—}001098818.1→NP_{—}001092288.1); PDE4D1 (NM_{—}001197222.1→NP_{—}001184151.1); PDE4D2 (NM_{—}001197221.1→NP_{—}001184150.1); PDE4D3 (NM_{—}006203.4→NP_{—}006194.2); PDE4D4 (NM_{—}001104631.1→NP_{—}001098101.1); PDE4D5 (NM_{—}001197218.1→NP_{—}001184147.1); PDE4D6 (NM_{—}001197223.1→NP_{—}001184152.1); PDE4D7 (NM_{—}001165899.1→NP_{—}001159371.1); PDE4D8 (NM_{—}001197219.1→NP_{—}001184148.1); PDE5A (NM_{—}001083.3→NP_{—}001074.2 (isoform 1); NM_{—}033430.2→NP_{—}236914.2 (isoform 2); NM_{—}033437.3→NP_{—}246273.2 (isoform 3)); PDE7A (NM_{—}002603.2→NP_{—}002594.1 (isoform a); NM_{—}002604.2→NP_{—}002595.1 (isoform b)); PDE7B (NM_{—}018945.3→NP_{—}061818.1); PDE8A (NM_{—}002605.2→NP_{—}002596.1 (isoform 1); NM_{—}173454.1→NP_{—}775656.1 (isoform 2)); PDE8B (NM_{—}003719.3→NP_{—}003710.1 (isoform 1); NM_{—}001029854.2→NP_{—}001025025.1 (isoform 2); NM_{—}001029851.2→NP_{—}001025022.1 (isoform 3); NM_{—}001029853.2→NP_{—}001025024.1 (isoform 4); NM_{—}001029852.2→NP_{—}001025023.1 (isoform 5)).

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mitedu/pubint/http://iona.wi.mit.edu/siRNAext/.”

c. Epoxygenated Fatty Acids

Administration of a PDEi increases levels of epoxygenated fatty acids (e.g., in blood, plasma, serum), usually without also increasing or only minimally increasing levels of dihydroxy-fatty acid. Exemplary epoxygenated fatty acids include cis-epoxyeicosantrienoic acids (“EETs”), epoxides of linoleic acid, epoxides of eicosapentaenoic acid (“EPA”) or epoxides of docosahexaenoic acid (“DHA”), or a mixture thereof.

i. EETs

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into dihydroxyeicosatrienoic acids (“DHETs”) is reduced. EETs that can be increased by administration of PDEi include 14,15-EET, 8,9-EET and 11,12-EET, and 5,6 EETs.

ii. Other Epoxygenated Fatty Acids

Exemplary epoxygenated fatty acids increased by administration of PDEi include epoxides of linoleic acid, eicosapentaenoic acid (“EPA”) and docosahexaenoic acid (“DHA”). See, e.g., Table 1.

Cytochrome P450 (“CYP450”) metabolism produces cis-epoxydocosapentaenoic acids (“EpDPEs”) and cis-epoxyeicosatetraenoic acids (“EpETEs”) from docosahexaenoic acid (“DHA”) and eicosapentaenoic acid (“EPA”), respectively. These epoxides are known endothelium-derived hyperpolarizing factors (“EDHFs”). These EDHFs, and others yet unidentified, are mediators released from vascular endothelial cells in response to acetylcholine and bradykinin, and are distinct from the NOS— (nitric oxide) and COX-derived (prostacyclin) vasodilators. Overall cytochrome P450 (CYP450) metabolism of polyunsaturated fatty acids produces epoxides, such as EETs, which are prime candidates for the active mediator(s). 14(15)-EpETE, for example, is derived via epoxidation of the 14,15-double bond of EPA and is the ω-3 homolog of 14(15)-EpETrE (“14(15)EET”) derived via epoxidation of the 14,15-double bond of arachidonic acid.

Like EETs, the epoxides of EPA and DHA are substrates for sEH. The epoxides of EPA and DHA are produced in the body at low levels by the action of cytochrome P450s. Endogenous levels of these epoxides can be maintained or increased by the administration of sEHI. However, the endogeous production of these epoxides is low and usually occurs in relatively special circumstances, such as the resolution of inflammation.

EPA has five unsaturated bonds, and thus five positions at which epoxides can be formed, while DHA has six. The epoxides of EPA are typically abbreviated and referred to generically as “EpETEs”, while the epoxides of DHA are typically abbreviated and referred to generically as “EpDPEs”. The specific regioisomers of the epoxides of each fatty acid are set forth in the following Table:

TABLE 1

A. Regioisomers of Eicosapentaenoic acid (“EPA”) epoxides:
1. Formal name: (±)5(6)- epoxy- 8Z, 11Z, 14Z, 17Z-
eicosatetraenoic acid,
Synonym 5(6)- epoxy Eicosatetraenoic acid
Abbreviation 5(6)- EpETE
2. Formal name: (±)8(9)- epoxy- 5Z, 11Z, 14Z, 17Z -
eicosatetraenoic acid,
Synonym 8(9)- epoxy Eicosatetraenoic acid
Abbreviation 8(9)- EpETE
3. Formal name: (±)11(12)- epoxy- 5Z, 8Z, 14Z, 17Z -
eicosatetraenoic acid,
Synonym 11(12)- epoxy Eicosatetraenoic acid
Abbreviation 11(12)- EpETE
4. Formal name: (±)14(15)- epoxy- 5Z, 8Z, 11Z, 17Z-
eicosatetraenoic acid,
Synonym 14(15)- epoxy Eicosatetraenoic acid
Abbreviation 14(15)- EpETE
5. Formal name: (±)17(18)- epoxy- 5Z, 8Z, 11Z, 14Z-
eicosatetraenoic acid,
Synonym 17(18)- epoxy Eicosatetraenoic acid
Abbreviation 17(18)- EpETE
B. Regioisomers of Docosahexaenoic acid (“DHA”) epoxides:
1. Formal name: (±) 4(5)- epoxy- 7Z, 10Z, 13Z, 16Z, 19Z -
docosapentaenoic acid,
Synonym 4(5)- epoxy Docosapentaenoic acid
Abbreviation 4(5)- EpDPE
2. Formal name: (±) 7(8)- epoxy- 4Z, 10Z, 13Z, 16Z, 19Z -
docosapentaenoic acid,
Synonym 7(8)- epoxy Docosapentaenoic acid
Abbreviation 7(8)- EpDPE
3. Formal name: (±)10(11)- epoxy- 4Z, 7Z, 13Z, 16Z, 19Z -
docosapentaenoic acid,
Synonym 10(11)- epoxy Docosapentaenoic acid
Abbreviation 10(11)- EpDPE
4. Formal name: (±)13(14)- epoxy- 4Z, 7Z, 10Z, 16Z, 19Z -
docosapentaenoic acid,
Synonym 13(14)- epoxy Docosapentaenoic acid
Abbreviation 13(14)- EpDPE
5. Formal name: (±) 16(17)- epoxy- 4Z, 7Z, 10Z, 13Z, 19Z -
docosapentaenoic acid,
Synonym 16(17)- epoxy Docosapentaenoic acid
Abbreviation 16(17)- EpDPE
6. Formal name: (±) 19(20)- epoxy- 4Z, 7Z, 10Z, 13Z, 16Z -
docosapentaenoic acid,
Synonym 19(20)- epoxy Docosapentaenoic acid
Abbreviation 19(20)- EpDPE

d. Soluble Epoxide Hydrolase Inhibitors (sEHi)

PDEi and sEHi cooperatively increase the levels of epoxygenated fatty acids in a subject. Therefore, in some embodiments, the PDEi is co-administered with an sEHi. One or both of the sEHi and the PDEi can be administered in a subtherapeutic dose.

Scores of sEH inhibitors are known, of a variety of chemical structures. Derivatives in which the urea, carbamate or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include:

12-(3-Adamantan-1-yl-ureido)dodecanoic acid (AUDA),

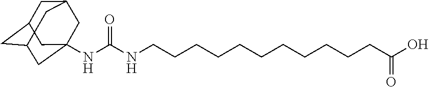 [SMILES: O=C(O)CCCCCCCCCCCNC(=O)NC12CC3CC(CC(C3)C1)C2]

12-(3-Adamantan-1-yl-ureido)dodecanoic acid butyl ester (AUDA-BE),

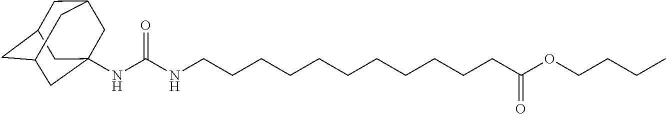 [SMILES: CCCCOC(=O)CCCCCCCCCCCNC(=O)NC12CC3CC(CC(C3)C1)C2]

Adamantan-1-yl-3-{5-[2-(2-ethoxyethoxy)ethoxy]pentyl}urea (compound 950, also referred to herein as “AEPU”), and

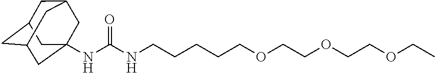 [SMILES: CCOCCOCCOCCCCCNC(=O)NC12CC3CC(CC(C3)C1)C2]

Another preferred group of inhibitors are piperidines. The following Table 2 sets forth some exemplar piperidines and their ability to inhibit sEH activity, expressed as the amount needed to reduce the activity of the enzyme by 50% (expressed as “IC₅₀”).

TABLE 2
IC₅₀ values for selected alkylpiperidine-based sEH inhibitors

| | | 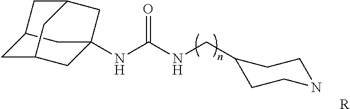 | | | |
|---|---|---|---|---|---|
| | | n = 0 | | n = 1 | |
| | | | IC₅₀ | | IC₅₀ |
| | | Compound | (μM)^{a} | Compound | (μM)^{a} |
| R: | H | I | 0.30 | II | 4.2 |
| | 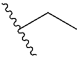 [SMILES: *CC] | 3a | 3.8 | 4.a | 3.9 |
| | 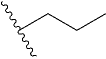 [SMILES: *CCC] | 3b | 0.81 | 4b | 2.6 |
| | 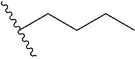 [SMILES: *CCCC] | 3c | 1.2 | 4c | 0.61 |
| | 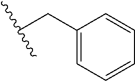 [SMILES: *Cc1ccccc1] | 3d | 0.01 | 4d | 0.11 |

^{a}As determined via a kinetic fluorescent assay.

A number of other sEH inhibitors which can be used in the methods and compositions of the invention are set forth in co-owned applications PCT/US2008/072199, PCT/US2007/006412, PCT/US2005/038282, PCT/US2005/08765, PCT/US2004/010298 and U.S. Published Patent Application Publication 2005/0026844, each of which is hereby incorporated herein by reference in its entirety for all purposes.

U.S. Pat. No. 5,955,496 (the '496 patent) also sets forth a number of sEH inhibitors which can be used in the methods of the invention. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH. These compounds also act as steric mimics of the corresponding epoxides of other fatty acids.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of sEH inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty sEH inhibitors of Formula 2, which were shown to inhibit human sEH at concentrations as low as 0.1 μM. Any particular sEH inhibitor can readily be tested to determine whether it will work in the methods of the invention by standard assays. Esters and salts of the various compounds discussed above or in the cited patents, for example, can be readily tested by these assays for their use in the methods of the invention.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the various uses of the invention contemplate inhibition of sEH over differing periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the sEH inhibitor used in the methods taught herein is a “soft drug.” Soft drugs are compounds of biological activity that are rapidly inactivated by enzymes as they move from a chosen target site. EETs and simple biodegradable derivatives administered to an area of interest may be considered to be soft drugs in that they are likely to be enzymatically degraded by sEH as they diffuse away from the site of interest following administration. Some sEHI, however, may diffuse or be transported following administration to regions where their activity in inhibiting sEH may not be desired. Thus, multiple soft drugs for treatment have been prepared. These include but are not limited to carbamates, esters, carbonates and amides placed in the sEHI, approximately 7.5 angstroms from the carbonyl of the central pharmacophore. These are highly active sEHI that yield biologically inactive metabolites by the action of esterase and/or amidase. Groups such as amides and carbamates on the central pharmacophores can also be used to increase solubility for applications in which that is desirable in forming a soft drug. Similarly, easily metabolized ethers may contribute soft drug properties and also increase the solubility.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 500 μM. Inhibitors with IC₅₀s of less than 500 μM are preferred, with IC₅₀s of less than 100 μM being more preferred and, in order of increasing preference, an IC50 of 50 μM, 40 μM, 30 μM, 25 μM, 20 μM, 15 μM, 10 μM, 5 μM, 3 μM, 2 μM, 1 μM or even less being still more preferred. Assays for determining sEH activity are known in the art and described elsewhere herein.

Other means of inhibiting sEH activity or gene expression can also be used in the methods of the invention. For example, a nucleic acid molecule complementary to at least a portion of the human sEH gene can be used to inhibit sEH gene expression. Means for inhibiting gene expression using short RNA molecules, as discussed above.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). An exemplary amino acid sequence of human sEH (GenBank Accession No. L05779) and an exemplary nucleotide sequence encoding that amino acid sequence (GenBank Accession No. AAA02756) are set forth in U.S. Pat. No. 5,445,956. The nucleic acid sequence of human sEH is also published as GenBank Accession No. NM_{—}001979.4; the amino acid sequence of human sEH is also published as GenBank Accession No. NP_{—}001970.2. Software programs for predicting siRNA sequences to inhibit the expression of a target protein are commercially available and find use, as discussed above.

e. Formulation, Dosing and Scheduling

The PDEi and/or sEHi can be prepared and administered independently or together in a wide variety of oral, parenteral and aerosol formulations. In some preferred forms, compounds for use in the methods of the present invention can be administered by injection, that is, intravenously, intramuscularly, intracutaneously, subcutaneously, intradermally, topically, intraduodenally, or intraperitoneally, while in others, they are administered orally. Administration can be systemic or local, as desired. The PDEi and/or sEHi can also be administered by inhalation. Additionally, the PDEi and/or sEHi can be administered transdermally. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from an PDEi and/or sEHi, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J. Dermatol., 81:386-389 (1972). A number of topical formulations of analgesics, including capsaicin (e.g., Capsin®), so-called “counter-irritants” (e.g., Icy-Hot®, substances such as menthol, oil of wintergreen, camphor, or eucalyptus oil compounds which, when applied to skin over an area presumably alter or off-set pain in joints or muscles served by the same nerves) and salicylates (e.g. BenGay®), are known and can be readily adapted for topical administration of the PDEi and/or sEHi, by replacing the active ingredient or ingredient with an PDEi and/or sEHi. It is presumed that the person of skill is familiar with these various vehicles and preparations and they need not be described in detail herein.

The PDEi and/or sEHi can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the agents are in a cream. Typically, the cream comprises one or more hydrophobic lipids, with other agents to improve the “feel” of the cream or to provide other useful characteristics. In one embodiment, for example, a cream of the invention may contain 0.01 mg to 10 mg of sEHI, with or without one or more EETs, per gram of cream in a white to off-white, opaque cream base of purified water USP, white petrolatum USP, stearyl alcohol NF, propylene glycol USP, polysorbate 60 NF, cetyl alcohol NF, and benzoic acid USP 0.2% as a preservative. In the studies reported in the Examples, sEHI were mixed into a commercially available cream, Vanicream® (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the agent or agents are in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the agent is, or agents are, in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

A therapeutically effective amount of the PDEi and/or sEHi is employed in reducing, alleviating, relieving, ameliorating, preventing and/or inhibiting neuropathic pain. The dosage of the specific compound for treatment depends on many factors that are well known to those skilled in the art. They include for example, the route of administration and the potency of the particular compound.

Determination of an effective amount is well within the capability of those skilled in the art. Generally, an efficacious or effective amount of an PDEi and/or sEHi is determined by first administering a low dose or a small amount of either the PDEi and/or sEHi and then incrementally increasing the administered dose or dosages, adding a second medication as needed, until a desired effect of is observed in the treated subject with minimal or no toxic side effects. An exemplary dose of an sEHi or EET is from about 0.001 μM/kg to about 100 mg/kg body weight of the mammal. sEH inhibitors with lower IC50 concentrations can be administered in lower doses.

Efficacious doses of phosphodiesterase inhibitors and neurosteroids are also known in the art. The present invention utilizes doses that are equivalent or less, e.g., doses that are about 75%, 50% or 25% of a full dose, to those prescribed for these agents when they are not co-administered with a PDEi and/or a sEHi. See, e.g., Physicians' Desk Reference 2009 (PDR, 63rd Edition) by Physicians' Desk Reference, 2008, Thomson Reuters.

In some formulations, the PDEi and/or sEHi are embedded in a slow-release formulation to facilitate administration of the agents over time.

In another set of embodiments, the PDEi and/or sEHi are administered by delivery to the nose or to the lung. Intranasal and pulmonary delivery are considered to be ways drugs can be rapidly introduced into an organism. Devices for delivering drugs intranasally or to the lungs are well known in the art. The devices typically deliver either an aerosol of a therapeutically active agent in a solution, or a dry powder of the agent. To aid in providing reproducible dosages of the agent, dry powder formulations often include substantial amounts of excipients, such as polysaccharides, as bulking agents.

Detailed information about the delivery of therapeutically active agents in the form of aerosols or as powders is available in the art. For example, the Center for Drug Evaluation and Research (“CDER”) of the U.S. Food and Drug Administration provides detailed guidance in a publication entitled: “Guidance for Industry: Nasal Spray and Inhalation Solution, Suspension, and Spray Drug Products—Chemistry, Manufacturing, and Controls Documentation” (Office of Training and Communications, Division of Drug Information, CDER, FDA, July 2002). This guidance is available in written form from CDER, or can be found on the worldwide web at “fda.gov/cder/guidance/4234fnl.htm”. The FDA has also made detailed draft guidance available on dry powder inhalers and metered dose inhalers. See, Metered Dose Inhaler (MDI) and Dry Powder Inhaler (DPI) Drug Products—Chemistry, Manufacturing, and Controls Documentation, 63 Fed. Reg. 64270, (November 1998). A number of inhalers are commercially available, for example, to administer albuterol to asthma patients, and can be used instead in the methods of the present invention to administer the PDEi and/or sEHi to subjects in need thereof.

In some aspects of the invention, the PDEi and/or sEHi is dissolved or suspended in a suitable solvent, such as water, ethanol, or saline, and administered by nebulization. A nebulizer produces an aerosol of fine particles by breaking a fluid into fine droplets and dispersing them into a flowing stream of gas. Medical nebulizers are designed to convert water or aqueous solutions or colloidal suspensions to aerosols of fine, inhalable droplets that can enter the lungs of a patient during inhalation and deposit on the surface of the respiratory airways. Typical pneumatic (compressed gas) medical nebulizers develop approximately 15 to 30 microliters of aerosol per liter of gas in finely divided droplets with volume or mass median diameters in the respirable range of 2 to 4 micrometers. Predominantly, water or saline solutions are used with low solute concentrations, typically ranging from 1.0 to 5.0 mg/mL.

Nebulizers for delivering an aerosolized solution to the lungs are commercially available from a number of sources, including the AERx™ (Aradigm Corp., Hayward, Calif.) and the Acorn II® (Vital Signs Inc., Totowa, N.J.).

Metered dose inhalers are also known and available. Breath actuated inhalers typically contain a pressurized propellant and provide a metered dose automatically when the patient's inspiratory effort either moves a mechanical lever or the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978; and 4,896,832.

The formulations may also be delivered using a dry powder inhaler (DPI), i.e., an inhaler device that utilizes the patient's inhaled breath as a vehicle to transport the dry powder drug to the lungs. Such devices are described in, for example, U.S. Pat. Nos. 5,458,135; 5,740,794; and 5,785,049. When administered using a device of this type, the powder is contained in a receptacle having a puncturable lid or other access surface, preferably a blister package or cartridge, where the receptacle may contain a single dosage unit or multiple dosage units.

Other dry powder dispersion devices for pulmonary administration of dry powders include those described in Newell, European Patent No. EP 129985; in Hodson, European Patent No. EP 472598, in Cocozza, European Patent No. EP 467172, and in Lloyd, U.S. Pat. Nos. 5,522,385; 4,668,281; 4,667,668; and 4,805,811. Dry powders may also be delivered using a pressurized, metered dose inhaler (MDI) containing a solution or suspension of drug in a pharmaceutically inert liquid propellant, e.g., a chlorofluorocarbon or fluorocarbon, as described in U.S. Pat. Nos. 5,320,094 and 5,672,581.

Without further elaboration, it is believed that one skilled in the art can, using the preceding description, practice the present invention to its fullest extent.

### EXAMPLES

The following examples are offered to illustrate, but not to limit the claimed invention.

### Example 1

### Materials and Methods

Animals

This study was approved by the institutional UC Davis Animal Care and Use Committee. Male Sprague-Dawley rats weighing 250-300 gr were obtained from Charles River Laboratories Inc. (Wilmington Mass.) and maintained in UC Davis animal housing facilities with ad libitum water and food on a 12 hr:12 hr light-dark cycle. A subset of rats was a generous donation from Charles River Laboratories. Data were collected during the same time of day for all groups.

Chemicals

The sEH inhibitors AUDA (12-(3-adamantan-1-yl-ureido)-dodecanoic acid) and TPAU (1-trifluoromethoxyphenyl-3-(1-acetylpiperidin-4-yl) urea) and TUPS (1-(1-methylsulfonyl-piperidin-4-yl)-3-(4-trifluoromethoxy-phenyl)-urea) were synthesized as previously reported (P. D. Jones, H.-J. Tsai, Z. N. Do, C. Morisseau, B. D. Hammock, Bioorganic & Medicinal Chemistry Letters 16, 5212 (2006); C. Morisseau, Goodrow, M. H., Newman, J. W., Wheelock, C. E., Dowdy, D. L., Hammock, B. D., Biochemical Pharmacology 63, 1599 (2002)). Rolipram was purchased from Biomol International (Plymouth Meeting, Pa.). All other chemicals were obtained from Sigma-Aldrich (St. Louis, Mo.) or Fisher Scientific (Pittsburg, Pa.).

Pain Models

PGE₂ elicited pain was produced by administering PGE₂ intraplantarly into one hind paw of the rat. Animals were then followed for their nociceptive responses over time as described previously (B. Inceoglu et al., Life Sciences 79 2311 (2006)). Three different doses of PGE₂ (10, 30 and 100 ng/paw) were administered as detailed in figure legends.

### Behavioral Tests and Treatments

Pain related behavior was measured using Hargreaves, von Frey and Randall-Selitto tests as described earlier (B. Inceoglu et al., Life Sciences 79 2311 (2006); B. Inceoglu et al., Proc Natl Acad Sci USA. 105, 18901 (2008)). Briefly, animals were acclimated to the testing room and the instrument. Baseline measurements were taken three times with 1 minute intervals between each measurement. The mean responses of animals were converted to % values by taking the baseline of each animal as 100%. Data are presented as % change from each animal's baseline thermal withdrawal latency. Baseline thermal withdrawal latencies varied between 6-10 seconds. Thermal withdrawal latency was monitored over time as described (K. Hargreaves, Dubner, R., Brown, F., Flores, C., Joris, J., Pain 32, 77 (1988)). Triolein was used as vehicle (Adams Vegetable Oils, Inc., Arbuckle, Calif.).

For the PGE₂ elicited pain model, the procedure of Khasar et al. was followed with modifications (S. G. Khasar, T. Ho, P. G. Green, J. D. Levine, Neuroscience 62, 345 (1994)). Animals were placed on an elevated steel mesh screen and enclosed in acrylic chambers. Following the baseline von Frey mechanical withdrawal thresholds animals were given vehicle, sEHi, celecoxib (20 mg/kg) or dexamethasone (5 mg/kg) subcutaneously dissolved in PEG400 (Fisher Scientific, Pittsburg, Pa.). One hour after the drug or vehicle PGE₂ was administered into the plantar surface of one hind paw. Animals were then probed with a plastic tipped force transducer connected to an electronic von Frey analgesiometer (IITC Woodland Hills, Calif.) until a withdrawal response was elicited. The test was conducted using the ‘MH’ (maximum holding) mode giving readout of the highest force in grams applied to the paw. Baseline von Frey mechanical withdrawal thresholds varied between 70-90 grams of force. The mean responses of animals were converted to % values by taking the baseline of each animal as 100%. Data are presented as % change from each animal's baseline thermal withdrawal threshold. The Δ reduction in pain was calculated by subtracting the mean % response of the PGE₂+ vehicle treated animals from the % response of each PGE₂+sEHI treated animal.

For the measurement of acute nociceptive responses thermal withdrawal latency was measured as described above or the method of Randall-Selitto was followed using an electronic analgesiometer (IITC Woodland Hills, Calif.). Animals were manually restrained and hind paw was placed between the tapering tip and the flat surface of a handheld Randall-Selitto apparatus. Force was applied manually until withdrawal was elicited. The test was conducted using the ‘MH’ mode giving readout of the highest force in grams applied to the paw. Following baseline measurements compounds (rolipram, TPAU, TUPS and AUDA) were administered subcutaneously after dissolving in PEG400. For the PDEi experiments sEHi were given one hour prior to PDEi. The antagonists flucanozole (40 mg/kg), micanozole (40 mg/kg), finasteride (10 mg/kg) were dissolved in PEG400 and were given 45 minutes before PDEi by subcutaneous injection. Picrotoxin (250 μg/kg) was dissolved in 10% ethanol in saline and administered at the same time as sEHi or 45 min prior to PDEi. Baseline mechanical withdrawal thresholds in this test varied between 70-90 grams of force. In groups treated with the PDEi immediately following PDEi administration, animals were placed in acrylic chambers on a glass platform maintained at a temperature of 30±1° C. for thermal withdrawal latency measurements.

All drug administrations were done subcutaneously on the back of the animals away from limbs.

For the measurement of open field activity animals were placed in an acrylic chamber (40×40×20 cm length×width×height) divided into 100 cm² (10×10 cm) sections. The number of crossings were recorded when both hind paws crossed into a neighboring cell.

Sampling, Extraction, Analyses of Inhibitors and Eicosanoids

Blood samples for eicosanoid analysis were collected using a 24 Gauge i.v. catheter (BD Insyte Autoguard) from the tail vein. Blood was centrifuged, plasma was separated and frozen. All samples were stored at −80° C. until analyses. For the determination of brain inhibitor levels animals were sacrificed following inhibitor administration by cardiac puncture while under deep isoflurane anesthesia. Animals were perfused using cold saline to remove traces of blood from brain tissue. The brains were removed following decapitation and frozen on dry ice. The blood and brain levels of TPAU were determined as described previously (B. Inceoglu et al., Proc Natl Acad Sci USA. 105, 18901 (2008)). Briefly, a small (˜50 mg) amount of the prefrontal cortex was removed and extracted three times with ethyl acetate containing the internal standard compound 869 (1-adamantan-1-yl-345-butoxy-pentyl)-urea, 250 ng/ml). The supernatants of three consecutive extractions were pooled and dried before resuspending in 50 μl of methanol. This sample was injected into an HPLC system. The separation was carried out by applying a linear solvent gradient from 10 to 100% ACN in 10 min. The separation module was connected to a Quattro Premier triple-quadrupole mass spectrometer (Waters, Milford, Mass.). The LC-ESI-MS/MS instrument was operated in positive electrospray ionization mode with selected reaction monitoring (SRM). The following transitions were monitored: m/z 337.3>160 for compound 869, and 346.3>169.4 for TPAU. Ionization parameters were same as described previously set to a capillary voltage of 1 kV, cone voltage of 25 V, source temperature of 110° C., desolvation temperature of 300° C. and desolvation gas flow of 645 l/hr.

Oxylipin analyses were carried out as described by Yang et al. with minor modifications (J. Yang, K. Schmelzer, K. Georgi, B. D. Hammock, Analytical Chemistry 81, 8085 (2009)). Briefly, an internal standard solution containing deuterated standards was added into the samples. This was followed by extraction of the analytes on a preconditioned solid phase extraction column (60 mg waters Oasis-HLB, Waters, Milford, Mass.). The eluted samples were evaporated to dryness and reconstituted in 50 μl of methanol. A 5 μL aliquot of the reconstructed sample solution was directly analyzed by LC-ESI-MS/MS. Separation was carried out on a Agilent 1200 SL LC system (Palo Alto, Calif.), utilizing a Agilent Zorbax Eclipse Plus C-18 reversed phase column (2.1×150 mm, 1.8 μM particle size) using a gradient of 0.1% acetic acid as solvent A and 80/15/0.1 acetonitrile/methanol/acetic acid as solvent B. The oxylipins were separated within 21 minutes using a using a similar gradient as described by Yang et al. (J. Yang, K. Schmelzer, K. Georgi, B. D. Hammock, Analytical Chemistry 81, 8085 (2009)). The column was reconditioned for 3.4 min at 35% solvent B before the next sample was introduced.

The detection was carried out using a 4000 QTRAP instrument (Applied Biosystems, Foster City, Calif.) operating in negative ion mode as previously described (J. Yang, K. Schmelzer, K. Georgi, B. D. Hammock, Analytical Chemistry 81, 8085 (08/28/, 2009)) by monitoring the following SRM transitions: 9(10)-EpOME (m/z 295/171), 9,10-DiHOME (m/z 313/201), 12(13)-EpOME (m/z 295/195), 12,13-DiHOME (m/z 313/183), 8(9)-EpETrE (m/z 319/167), 8,9-DiHETrE (m/z 337/127), 11(12)-EpETrE (m/z 319/167), 11,12-DiHETrE (m/z 337/167), 14(15)-EpETrE (m/z 319/219), 14,15-DiHETrE (m/z 337/207), 8(9)-EpETE (m/z 317/127), 8,9-DiHETE (m/z 335/127), 11(12)-EpETE (m/z 317/167), 11,12-DiHETE (m/z 335/167), 14(15)-EpETE (m/z 317/207), 14,15-DiHETE (m/z 335/207), 17(18)-EpETE (m/z 317/215), 17,18-DiHETE (m/z 335/247), 10(11)-EpDPE (m/z 343/153), 10,11-DiHDPE (m/z 361/153), 13(14)-EpDPE (m/z 343/193), 13,14-DiHDPE (m/z 361/193), 16(17)-EpDPE (m/z 343/233), 16,17-DiHDPE (m/z 361/233), 19(20)-EpDPE (m/z 343/241) and 19,20-DiHDPE (m/z 361/273).

Enzyme Assays and Synthesis of Inhibitors

Potency of the sEHi were determined using a modified procedure as described previously (B. Inceoglu et al., Proc Natl Acad Sci USA. 105, 18901 (2008); R. N. Wixtrom, B. D. Hammock, Analytical Biochemistry 174, 291 (1988)). Recombinant enzymes cloned from mouse, rat and human were expressed by a baculovirus expression system followed by purification through an affinity chromatography step (R. N. Wixtrom, B. D. Hammock, Analytical Biochemistry 174, 291 (1988)). Pierce BCA assay was used to quantify protein amounts. The concentration that leads to the inhibition of half of the enzyme activity by an inhibitor was assigned as the IC₅₀ for that compound. Potency on recombinant sEH from the mouse, rat and human were determined using a fluorescent substrate, cyano(2-methoxynaphthalen-6-yl)methyl trans-(3-phenyl-oxyran-2-yl) methyl carbonate (P. D. Jones et al., Analytical Biochemistry 343, 66 (2005)). Rolipram was tested for inhibitory activity using recombinant human and rat enzymes by incorporating 10 and 100 μM of rolipram into the sEH assay. No inhibition was observed by rolipram. Each IC₅₀ experiment included at least five different concentrations of inhibitor determined in triplicates. For rolipram only two concentrations were used. Inhibitors of sEH were synthesized, purified and characterized in our laboratory as described previously (P. D. Jones, H.-J. Tsai, Z. N. Do, C. Morisseau, B. D. Hammock, Bioorganic & Medicinal Chemistry Letters 16, 5212 (2006); C. Morisseau, B. D. Hammock, in Techniques for analysis of chemical biotransormation. Current Protocols in Toxicology J. S. Bus, Costa, L. G., Hodgson, E., Lawrence, D. A. and Reed, D. J., Ed. (John Wiley & Sons, New Jersey, 2007) pp. 4.23.1 180).

Statistical Analyses

Data were analyzed by ANOVA followed by Tukey's post hoc test for between group comparisons using the SPSS analysis package (SPSS, Chicago, Ill.). Results are depicted as mean±SEM. Regression equations were used for the calculation of IC₅₀s.

### Example 2

### sEHi and PDEi Modulate Epoxygenated Fatty Acid Levels

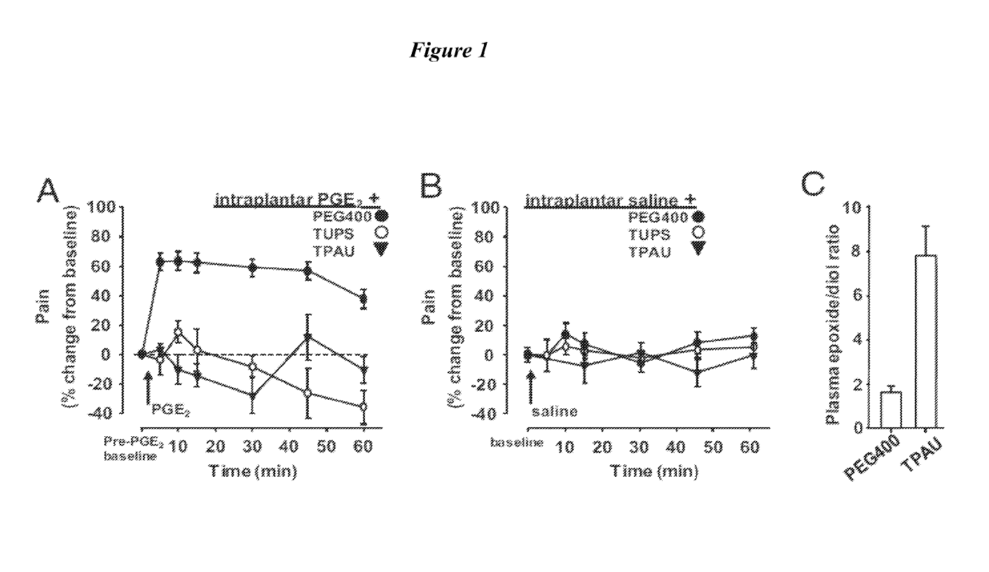

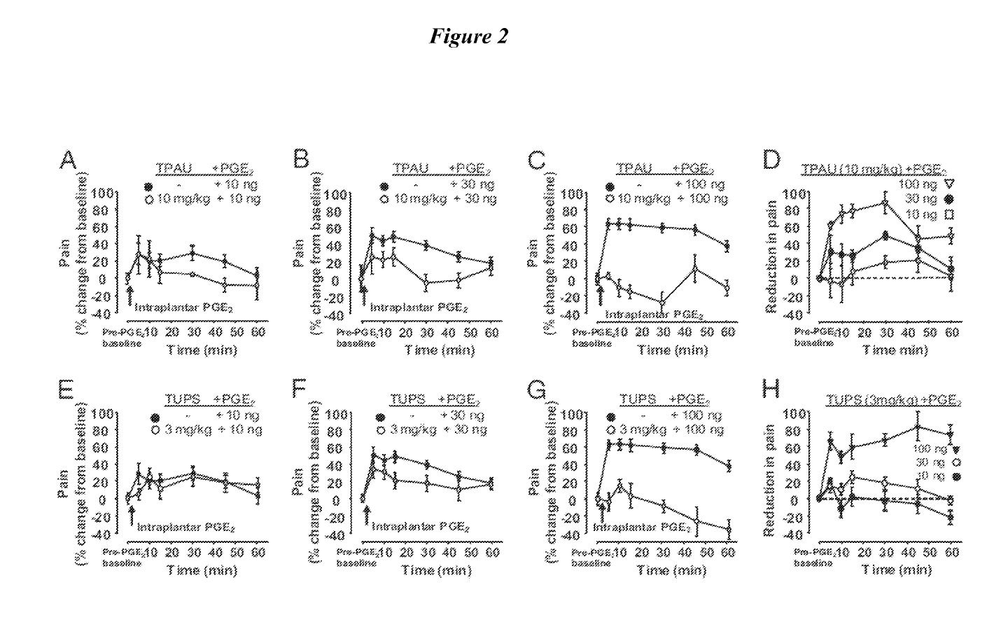

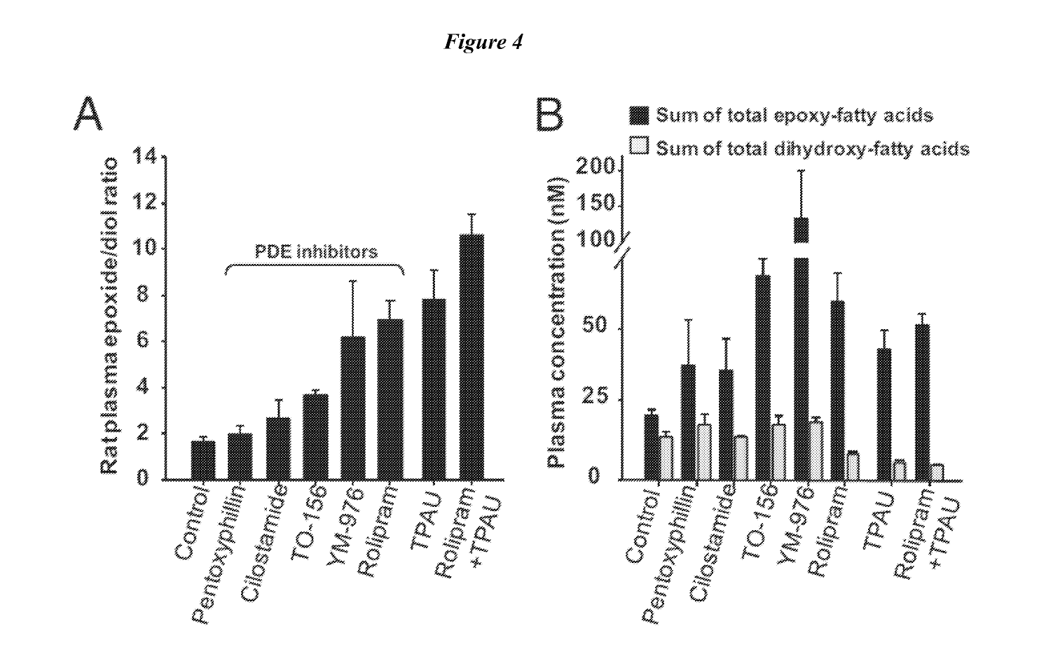

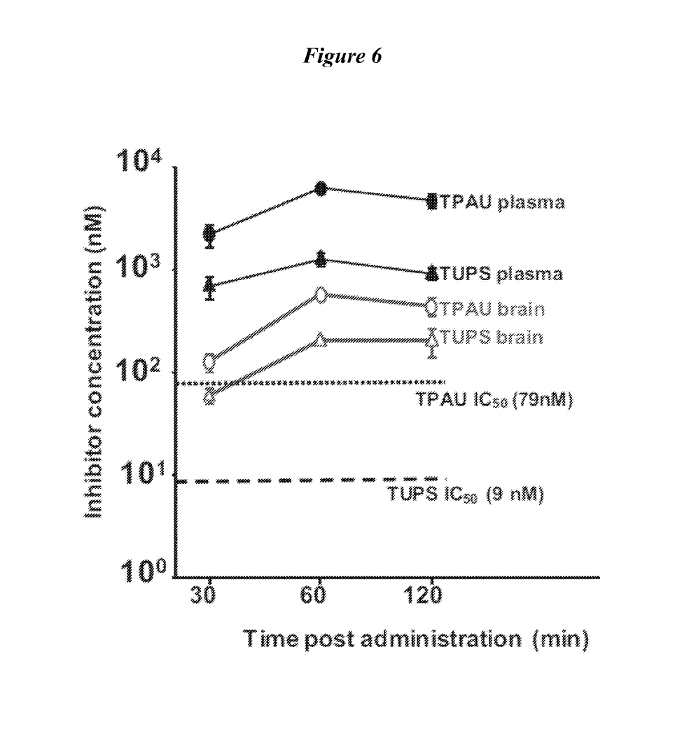

The sEH tightly controls the levels of the natural EFA (Spector and Norris, Am J Physiol Cell Physiol. (2007) 292(3):C996-1012). Consistent with the structural diversity of the EFA, in vivo inhibition of sEH results in a variety of beneficial effects in disease models including anti-hypertensive, anti-inflammatory and pain blocking activities (Imig, et al., Hypertension (2002) 39(2 Pt 2):690-4); Inceoglu et al., Life Sciences (2006) 79(24):2311-9; Schmelzer et al., Proc Natl Acad Sci USA. (2006) 103(37):13646-51; Inceoglu, et al., Prostaglandins & Other Lipid Mediators (2007) 82(1-4):42-9; Terashvili et al., J Pharmacol Exp Ther. (2008) 326(2):614-22). However the mechanisms of action of the EFA are largely unknown. The sEH, a mainly cytosolic enzyme (Morisseau and Hammock, Annual Review of Pharmacology and Toxicology (2005) 45:311-33) is expressed in the central nervous system (CNS) (Sura, et al., J Histochem Cytochem. (2008) 56(6):551-9); though its role in pain control is unexpected and is different from known agents (FIGS. 1 and 2). Specifically, sEHi block pain produced by the proinflammatory prostanoid PGE₂, a product of cyclooxygenase (FIG. 1A), although this pain is impervious to reversal by conventional anti-inflammatory agents, NSAIDs (non steroidal anti-inflammatory agents) (Khasar, et al., Neuroscience (1994) 62(2):345-50) selective cyclooxygenase inhibitors and steroids, (FIG. 4). Resembling the NSAIDs, the sEHi do not have effects on acute pain thresholds (i.e., in the absence of a persistent pain state) even at doses over 30 fold greater than that needed to reduce existing pain (FIG. 1B). However, in vivo, sEHi penetrate to the brain (FIG. 6) and increase the plasma and tissue epoxy to dihydroxy-fatty acid ratios, an outcome of inhibiting sEH, regardless of the disease status of the animals.

Elevation of EFAs Blocks Noninflammatory Pain.

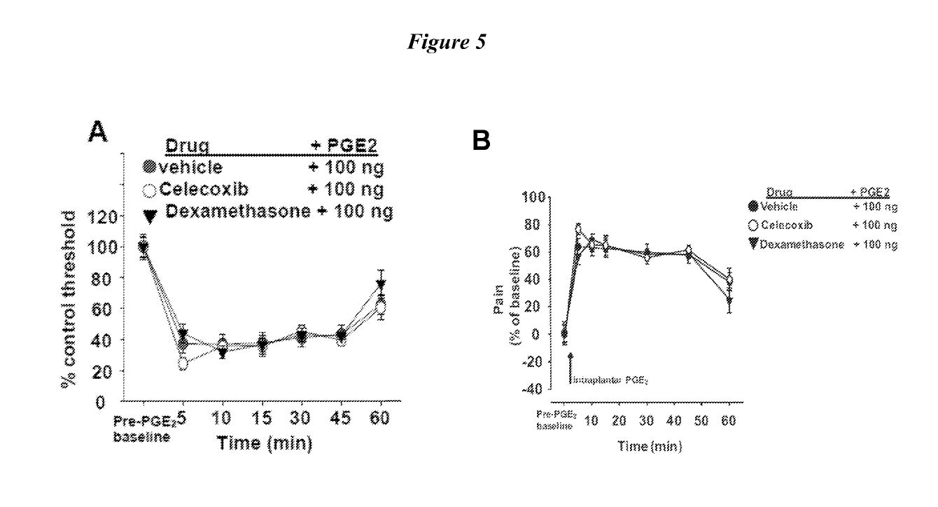

Inhibitors of sEH reduce inflammatory pain, consistent with other reports suggesting that EFAs are anti-inflammatory molecules (Node, et al. (1999) Science 285:1276-1279; Morisseau, et al. (2010) J Lipid Res 51:3481-3490; Inceoglu, et al. (2008) Proc Natl Acad Sci USA 105:18901-18906). However, sEH inhibitors (sEHIs) also block neuropathic pain in diabetic animals (Inceoglu, et al. (2008) Proc Natl Acad Sci USA 105:18901-18906). To test whether sEHIs are antinociceptive independent from reducing inflammation, pain was induced by using prostaglandin E2 (PGE₂). This model involving direct application of PGE₂ is devoid of a major inflammatory component and therefore pain elicited by this COX product is impervious to reversal by most drugs targeting the arachidonic acid cascade, including nonsteroidal anti-inflammatory drug (NSAIDs) (Khasar, et al., (1994) Neuroscience 62:345-350), selective cyclooxygenase inhibitors, and steroids (FIG. 5). In contrast to these agents, the sEHIs effectively blocked pain elicited by PGE₂ (FIG. 1A), consistent with the conclustion that sEHIs reduce pain independent from their anti-inflammatory activity.

EFAs Act in a Pain-Dependent Manner.

The sEHIs stabilize and thus elevate antinociceptive and anti-inflammatory EFAs whereas the NSAIDs reduce pain by blocking the synthesis of proinflammatory molecules. Unlike narcotic agents that are analgesic even in the absence of pain, the sEHIs have minimal effects on basal acute pain thresholds (FIG. 1B and FIG. 6) even at doses more than 30 fold greater than that needed to reduce existing pain (Inceoglu, et al. (2008) Proc Natl Acad Sci USA 105:18901-18906). Such sEHI levels elevate the EFAs and simultaneously decrease the inactive degradation products dihydroxy-fatty acids (FAs) in plasma and tissues regardless of the disease status of the animals (FIG. 1C and Table 3). Therefore, elevation of the EFA levels per se does not appear to be sufficient to modulate pain-related behavior.

It was tested if the pain-blocking effects of sEHIs require factor(s) in addition to elevated EFAs. Such factor(s) would be endogenously generated during the pain response. Thus, the effect of the intensity of the pain state on the efficacy of sEHIs was evaluated. Pain elicited by a series of increasing amounts of PGE₂ in the presence of a constant dose of sEHI was quantified (FIG. 2 A-C and E-G). Although sEHIs effectively blocked intense pain elicited by the high dose of PGE₂ (100 ng per paw), their efficacy diminished proportionally with lower doses of PGE₂ (FIGS. 2 D and H). A major EFA, 14,15-EpETre, was recently reported to have no interaction with D- or E-prostanoid receptors (Behm, et al., (2009) J Pharmacol Exp Ther 328: 231-239). Given that EFAs do not seem to be antagonists of the E-prostanoid receptors, these observations are consistent with the conclusion that the pain-reducing effects of sEHI and EFAs are pain activity-dependent.

Because PGE₂-elicited E-prostanoid receptor activation leads to adenylate cyclase activation, generation of cAMP and subsequently to pain (P. F. Vonvoigtlander, E. G. Losey, Brain Research 128, 275 (1977)), it was determined that cAMP is an important chemical mediator which when present dramatically increases the ability of sEHi to reduce pain. Therefore, the levels of cAMP were modulated using rolipram, a PDE inhibitor (PDEi). However, given that intracellular cAMP is increased by inflammation and is painful (P. F. Vonvoigtlander, E. G. Losey, Brain Research 128, 275 (1977); S. Burstein, G. Gagnon, S. A. Hunter, D. V. Maudsley, Prostaglandins 13, 41 (1977); U. Zor, Toshio Kaneko, Herman P. G. Schneider, Samuel M. McCann, Irene P. Lowe, Gail Bloom, Barbara Borland, and James B. Field, Proc Natl Acad Sci USA. 63, 918 (1969); Song, et al., J Neurophysiol. (2006) 95(1):479-92), in the following experiments healthy rats without inflammation or neuropathy were used and acute pain-related behavior measured as withdrawal responses to thermal and mechanical stimuli was monitored.

This allowed testing of the effects of a constant dose of sEHI in a paradigm that is independent of an underlying pain status but in which cAMP is artificially elevated by using rolipram, a phosphodiesterase (PDE) 4 inhibitor (PDEi). Rolipram is reported to enhance existing pain when administered locally (Taiwo, et al., (1991) Neuroscience 44:131-135). Here, systemic administration of rolipram itself was effective in elevating pain thresholds (FIG. 3). Strikingly, sEHIs that were devoid of effect in healthy animals, when co-administered with the PDEi, largely blunted pain-related behavior, displaying an opioid-like analgesic effect (FIG. 3). These findings argue that EFAs and sEHI block pain by positively interacting with a cAMP-dependent pathway.

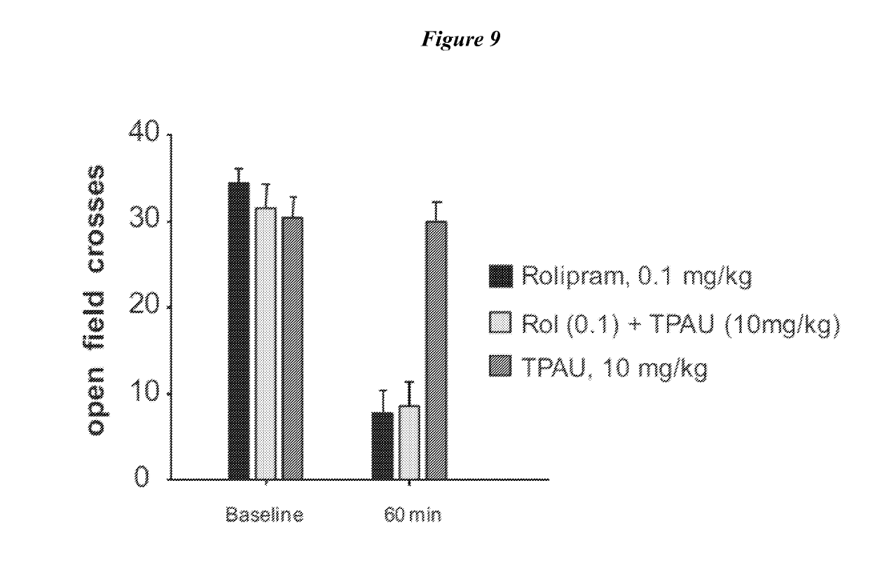

Although rolipram seemed to block acute nociceptive pain behavior in our experiments, it also led to decreased mobility as reported (Wachtel (1982) Psychopharmacology (Berl) 77:309-316). In contrast, the sEHI alone did not reduce mobility (FIG. 9). At low doses of rolipram at which motor depressant effects are not maximal, a synergistic elevation in pain thresholds was evident if sEHI was co-administered (FIG. 3). Given the depressant effects of rolipram, this could be a result of a synergistic increase in motor depression when sEHI and PDEi were administered. However, no synergy was observed in motor depression when sEHI and PDEi were administered (FIG. 9). Strikingly, 2 and 4 h after treatments, rolipram was devoid of effect on withdrawal latency whereas sEHI plus PDEi treatment was highly effective in attenuating pain-related behavior.

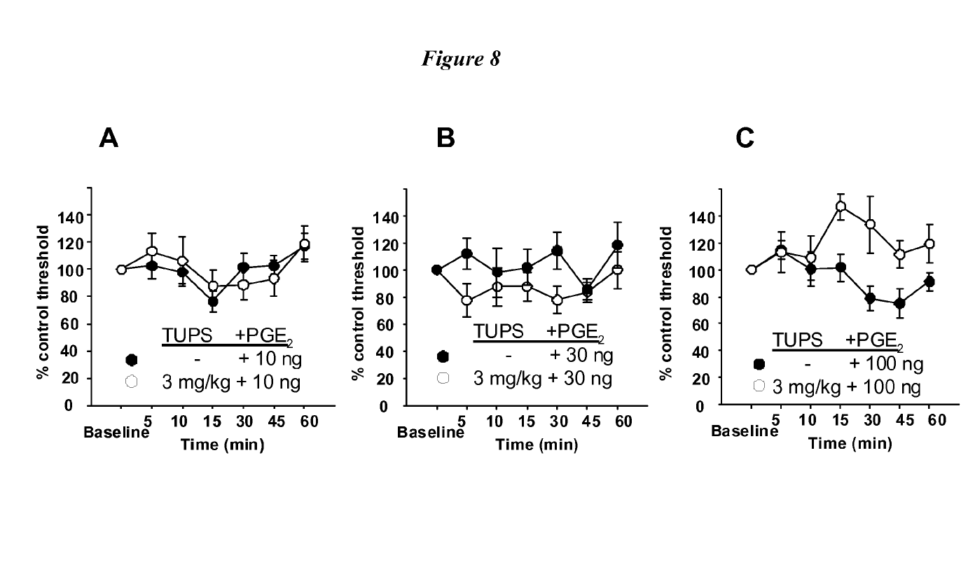

Although rolipram was shown to block pain, it also lead to decreased mobility (H. Wachtel, Psychopharmacology 77, 309 (1982)). In contrast, the sEHi do not reduce mobility (FIG. 8). Moreover, the combination of sEHi+PDEi did not result in further decrease in mobility than that already observed for the PDEi. The lack of effect of sEHi in the absence of pain allowed us to give constant high doses of sEHi to strongly inhibit the sEH in the preceding experiments. These doses led to plasma inhibitor levels >200 fold higher than the IC₅₀ determined for the recombinant rat sEH and increased the fatty acid epoxide/diol ratio in the plasma, an indication of in vivo target engagement (FIG. 1D). Although epoxide hydration is the major route of epoxy-fatty acid degradation, when it is inhibited other pathways of metabolism rapidly remove EFA from circulation leading to a net increase of only several fold (Spector and Norris, Am J Physiol Cell Physiol. (2007) 292(3):C996-1012).

Inhibitors of PDE and sEH Have Distinct Pharmacological Actions but both Modulate Bioactive Lipids in Plasma.

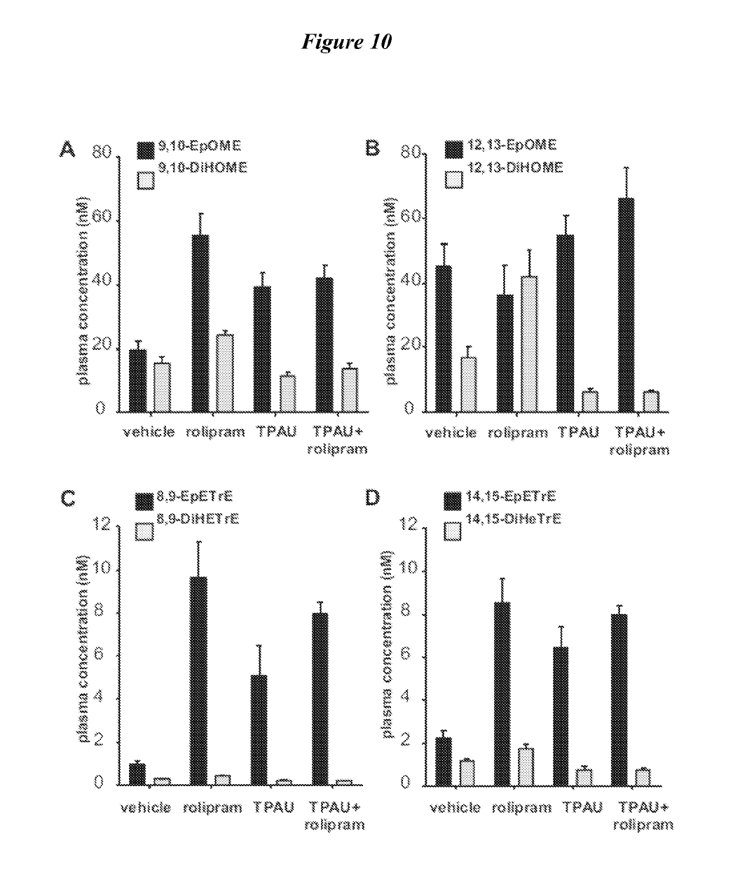

While quantifying plasma fatty acid epoxide/diol ratios in sEHI treated animals as a quantitative measure of target engagement, it was included the plasma of PDEi-treated animals as negative control. It was unexpected to find that rolipram was highly effective in elevating absolute quantity of EFAs and fatty acid epoxide/diol ratios in plasma (FIG. 4). Indeed, other selective PDEis also led to elevation of EFAs (FIG. 4). Remarkably, the sEHI and PDEi modulated the EFAs distinctly, with sEHI elevating EFAs and expectedly reducing the levels of corresponding dihydroxy-FAs whereas PDEi primarily elevated EFAs and displayed minimal effects on dihydroxy-FAs (FIG. 10 demonstrates exceptions). Consistent with the structural differences in sEHI and PDEi, rolipram lacked inhibitory activity on recombinant rat or human sEH (IC50>100 μM). Therefore, the increase in EFAs by PDEi is a physiological response. Accordingly, the PDEis are a new class of non-sEHI pharmacological agents that selectively boost EFAs without impinging on the dihydroxy-FA metabolites (Tables 3 and 4).

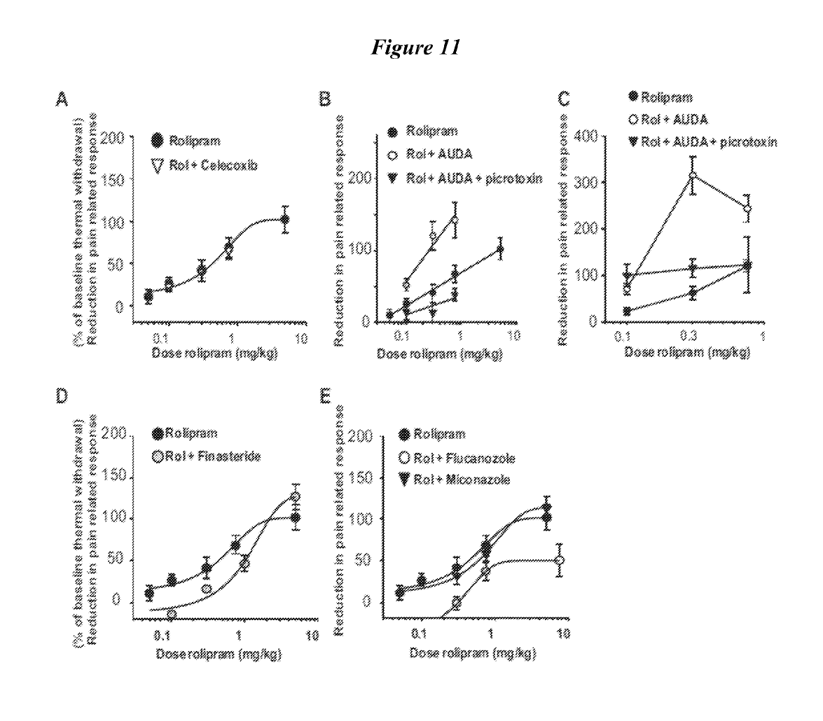

Despite this unanticipated overlap in the abilities of both classes of compounds to elevate the epoxide/diol ratio, the effects of the sEHI and co-administration of the sEHI with PDEi were clearly distinguishable from PDEi alone (FIG. 11). Specifically, the sEHI treatment in healthy animals elevated the epoxide/diol ratios but did not change pain-related behavior or mobility, whereas PDEi alone seemed to decrease pain-related behavior and depressed mobility. In contrast, co-administration of sEHI and PDEi produced an additive increase in the epoxy/diol fatty acid ratio in plasma while synergistically elevating the nociceptive pain thresholds.

Moreover, when the de novo synthesis of EFA was blocked by using a CNS permeable cytochrome P450 epoxygenase inhibitor, the PDEi produced analgesia was blocked in non-competitive and non-surmountable manner demonstrating that only a fraction of the analgesic effects is dependent on EFA (FIG. 11E). The CNS impermeable inhibitor completely lacked antagonistic effect (FIG. 11E). These data demonstrate that CNS mediated antinociceptive effects of PDEi prevail over peripherally mediated effects. At the same time, a considerable fraction of the PDEi's pain reducing effect seems to be mediated by EFA.

Overall, efficacy of sEHi against pain and production of profound analgesia when administered with PDEi are consistent with the conclusion that natural EFA cooperatively act with cAMP. These findings support earlier observations that EFA and sEH have important roles in disease or pain state modulated signaling. Concurrent inhibition of sEH and PDE provides a number of advantages, in particular, if used as post operative analgesics or during recovery from general anesthesia, when the transient somatosensory depressant effects of PDEi are desirable. Consequently, systemically delivered sEHi and sEHi+PDEi combinations find use in the clinic for inflammatory and painful conditions.

TABLE 3
Quantitative analysis (mean ± SEM) of endogenous oxylipin sEH substrates and
products in rat plasma following sEHI (TPAU) and rolipram administration

| | | | | | | | | | | | TPAU + | | |
|---|---|---|---|---|---|---|---|---|---|---|---|---|---|
| | | Control | | | | | | | | | Rolipram | | |
| Parent fatty acid | Metabolite oxylipin | nM | | SEM | Rolipram nM | | SEM | TPAU nM | | SEM | nM | | SEM |
| Linoleate | 9(10)-EpOME | 19.67 | | 2.41 | 55.27 | | 7.13 | 39.27 | | 4.44 | 42.00 | | 3.86 |
| C18:2 | 9,10-DiHOME | 15.52 | | 2.10 | 24.32 | | 1.43 | 11.51 | | 1.00 | 13.77 | | 1.69 |
| | Ratio | | 1.27 | | | 2.27 | | | 3.41 | | | 3.05 | |
| | 12(13)-EpOME | 45.07 | | 6.90 | 36.32 | | 9.08 | 54.70 | | 6.32 | 66.03 | | 9.47 |
| | 12,13-DiHOME | 16.96 | | 3.11 | 42.01 | | 8.24 | 6.34 | | 1.01 | 6.15 | | 0.57 |
| | Ratio | | 2.66 | | | 0.86 | | | 8.63 | | | 10.74 | |
| Arachidonate | 8(9)-EpETrE | 0.99 | | 0.15 | 9.63 | | 1.69 | 5.11 | | 1.36 | 7.93 | | 0.59 |
| C20:4 | 8,9-DiHETrE | 0.29 | | 0.03 | 0.45 | | 0.04 | 0.22 | | 0.03 | 0.23 | | 0.01 |
| | Ratio | | 3.37 | | | 21.25 | | | 23.30 | | | 34.41 | |
| | 11(12)-EpETrE | 2.43 | | 0.41 | 13.15 | | 2.23 | 10.45 | | 2.06 | 11.85 | | 0.81 |
| | 11,12-DiHETrE | 1.48 | | 0.19 | 1.67 | | 0.15 | 1.25 | | 0.20 | 1.18 | | 0.16 |
| | Ratio | | 1.64 | | | 7.88 | | | 8.37 | | | 10.03 | |
| | 14(15)-EpETrE | 2.24 | | 0.34 | 8.52 | | 1.14 | 6.45 | | 0.96 | 7.99 | | 0.40 |
| | 14,15-DiHETrE | 1.17 | | 0.11 | 1.74 | | 0.19 | 0.78 | | 0.16 | 0.77 | | 0.09 |
| | Ratio | | 1.91 | | | 4.89 | | | 8.31 | | | 10.35 | |
| Eicosapentanoate | 8(9)-EpETE | 0.13 | | 0.05 | 1.25 | | 0.26 | 0.85 | | 0.21 | 1.18 | | 0.18 |
| C20:5 | 8,9-DiHETE | 0.12 | | 0.04 | 0.07 | | 0.01 | 0.06 | | 0.01 | 0.04 | | 0.004 |
| | Ratio | | 1.02 | | | 18.18 | | | 14.01 | | | 28.27 | |
| | 11(12)-EpETE | 0.05 | | 0.02 | 0.93 | | 0.18 | 0.51 | | 0.09 | 0.78 | | 0.11 |
| | 11,12-DiHETE | 0.35 | | 0.04 | 0.20 | | 0.02 | 0.13 | | 0.01 | 0.13 | | 0.01 |
| | Ratio | | 0.14 | | | 4.72 | | | 4.02 | | | 6.19 | |
| | 14(15)-EpETE | 0.48 | | 0.11 | 0.84 | | 0.14 | 0.54 | | 0.06 | 0.76 | | 0.11 |
| | 14,15-DiHETE | 0.70 | | 0.07 | 0.28 | | 0.04 | 0.15 | | 0.02 | 0.12 | | 0.02 |
| | Ratio | | 0.68 | | | 2.94 | | | 3.68 | | | 6.34 | |
| | 17(18)-EpETE | 1.69 | | 0.31 | 1.76 | | 0.23 | 1.29 | | 0.15 | 1.35 | | 0.12 |
| | 17,18-DiHETE | 1.95 | | 0.21 | 0.77 | | 0.12 | 0.40 | | 0.05 | 0.34 | | 0.03 |
| | Ratio | | 0.86 | | | 2.30 | | | 3.20 | | | 3.96 | |
| Docosahexanoate | 10(11)-EpDPE | 2.24 | | 0.43 | 7.51 | | 1.33 | 5.62 | | 1.09 | 6.74 | | 0.64 |
| C22:6 | 10,11-DiHDPE | 0.50 | | 0.04 | 0.33 | | 0.03 | 0.23 | | 0.03 | 0.20 | | 0.02 |
| | Ratio | | 4.53 | | | 22.87 | | | 24.19 | | | 34.58 | |
| | 13(14)-EpDPE | 1.23 | | 0.21 | 3.88 | | 0.60 | 2.90 | | 0.53 | 3.42 | | 0.36 |
| | 13,14-DiHDPE | 0.64 | | 0.07 | 0.37 | | 0.03 | 0.36 | | 0.06 | 0.25 | | 0.02 |
| | Ratio | | 1.92 | | | 10.54 | | | 8.12 | | | 13.61 | |
| | 16(17)-EpDPE | 1.69 | | 0.14 | 3.93 | | 0.63 | 3.11 | | 0.50 | 3.49 | | 0.32 |
| | 16,17-DiHDPE | 1.53 | | 0.31 | 0.72 | | 0.08 | 0.50 | | 0.10 | 0.39 | | 0.03 |
| | Ratio | | 1.10 | | | 5.45 | | | 6.19 | | | 8.99 | |
| | 19(20)-EpDPE | 8.08 | | 0.75 | 7.46 | | 0.88 | 5.93 | | 0.82 | 5.63 | | 0.36 |
| | 19,20-DiHDPE | 5.30 | | 1.27 | 2.13 | | 0.17 | 1.64 | | 0.23 | 1.27 | | 0.06 |
| | Ratio | | 1.52 | | | 3.50 | | | 3.61 | | | 4.42 | |
| total | Epoxyeicosanoid | 86.0 | | | 150.4 | | | 136.7 | | | 159.2 | | |
| | Dihydroxyeicosanoid | 46.5 | | | 75.1 | | | 23.6 | | | 24.8 | | |
| | Ratio | | 1.85 | | | 2.0 | | | 5.80 | | | 6.41 | |

The oxylipins were quantified according to the methods and references. Drugs were administered s.c., and blood was taken 60 min following TPAU (10 mg/kg, n = 6), rolipram (1 mg/kg, n = 12), and TPAU/rolipram (10 and 1 mg/kg, respectively, n = 6). Below, oxylipins are grouped based on their parent molecules, linoleic acid, arachidonic acid, docosahexaenoic acid, and eicosapentaenoic acid (first column). The mean and SE (SEM) of the determined concentration (in nM) is presented. Ratio of epoxy/dihydroxy eicosanoids for each epoxide/diol pair is also shown. The SEM for ratios was omitted for clarity. The graphs presented in FIG. 3 include sum of ARA, docosahexaenoic acid, and eicosapentaenoic acid metabolites listed here. FIG. 6 shows plasma levels of TPAU at the time of sampling.

TABLE 4
Quantitative analysis of endogenous oxylipin sEH substrates and products in rat plasmafollowing PDE inhibitor administration

| | | Pentoxyphilline | | | Cilostamide | | | TO-156 | | | YM976 | | |
|---|---|---|---|---|---|---|---|---|---|---|---|---|---|
| | Metabolite | nM | | | nM | | | nM | | | nM | | |
| Parent fatty acid | oxylipin | n = 4 | | SEM | n = 8 | | SEM | n = 4 | | SEM | n = 4 | | SEM |
| Linoleate | 9(10)-EpOME | 59.92 | | 13.06 | 32.41 | | 19.84 | 34.27 | | 7.46 | 147.05 | | 82.37 |
| C18:2 | 9,10-DiHOME | 34.24 | | 4.51 | 29.53 | | 8.15 | 38.31 | | 4.50 | 37.07 | | 8.80 |
| | Ratio | | 1.75 | | | 1.10 | | | 0.89 | | | 3.97 | |
| | 12(13)-EpOME | 87.05 | | 22.35 | 51.82 | | 22.89 | 63.31 | | 10.39 | 159.31 | | 62.70 |
| | 12,13-DiHOME | 50.89 | | 3.87 | 37.98 | | 11.77 | 53.53 | | 6.43 | 57.32 | | 17.81 |
| | Ratio | | 1.71 | | | 1.36 | | | 1.18 | | | 2.78 | |
| Arachidonate | 8(9)-EpETrE | 6.41 | | 0.36 | 3.13 | | 1.43 | 3.66 | | 1.32 | 13.59 | | 7.73 |
| C20:4 | 8,9-DiHETrE | 0.54 | | 0.00 | 0.47 | | 0.07 | 0.49 | | 0.03 | 0.58 | | 0.11 |
| | Ratio | | 11.91 | | | 6.69 | | | 7.43 | | | 23.44 | |
| | 11(12)-EpETrE | 10.60 | | 0.59 | 4.44 | | 2.15 | 5.43 | | 2.10 | 22.80 | | 12.98 |
| | 11,12-DiHETrE | 1.77 | | 0.18 | 1.60 | | 0.37 | 1.89 | | 0.13 | 1.55 | | 0.20 |
| | Ratio | | 6.00 | | | 2.78 | | | 2.87 | | | 14.76 | |
| | 14(15)-EpETrE | 14.76 | | 0.74 | 7.71 | | 3.03 | 8.67 | | 2.49 | 26.50 | | 12.68 |
| | 14,15-DiHETrE | 1.73 | | 0.03 | 1.82 | | 0.28 | 1.74 | | 0.04 | 1.84 | | 0.28 |
| | Ratio | | 8.52 | | | 4.24 | | | 4.98 | | | 14.43 | |
| Eicosapentanoate | 8(9)-EpETE | 1.27 | | 0.24 | 0.74 | | 0.33 | 0.72 | | 0.29 | 2.74 | | 1.48 |
| C20:5 | 8,9-DiHETE | 0.30 | | 0.04 | 0.27 | | 0.10 | 0.20 | | 0.04 | 0.29 | | 0.03 |
| | Ratio | | 4.15 | | | 2.81 | | | 3.52 | | | 9.62 | |
| | 11(12)-EpETE | 0.80 | | 0.07 | 0.45 | | 0.21 | 0.50 | | 0.19 | 2.23 | | 1.29 |
| | 11,12-DiHETE | 0.45 | | 0.05 | 0.43 | | 0.11 | 0.35 | | 0.04 | 0.48 | | 0.08 |
| | Ratio | | 1.79 | | | 1.04 | | | 1.44 | | | 4.69 | |
| | 14(15)-EpETE | 0.69 | | 0.04 | 0.49 | | 0.21 | 0.55 | | 0.17 | 1.99 | | 0.94 |
| | 14,15-DiHETE | 1.06 | | 0.12 | 1.24 | | 0.32 | 0.82 | | 0.13 | 1.19 | | 0.11 |
| | Ratio | | 0.66 | | | 0.39 | | | 0.67 | | | 1.67 | |
| | 17(18)-EpETE | 3.08 | | 0.49 | 2.66 | | 0.64 | 2.30 | | 0.25 | 4.49 | | 1.12 |
| | 17,18-DiHETE | 2.81 | | 0.44 | 3.08 | | 0.67 | 1.74 | | 0.14 | 2.95 | | 0.29 |
| | Ratio | | 1.10 | | | 0.86 | | | 1.32 | | | 1.52 | |
| Docosahexanoate | 10(11)-EpDPE | 7.86 | | 0.88 | 4.06 | | 2.08 | 3.46 | | 1.40 | 20.16 | | 11.46 |
| C22:6 | 10,11-DiHDPE | 0.71 | | 0.10 | 0.66 | | 0.18 | 0.54 | | 0.05 | 0.82 | | 0.13 |
| | Ratio | | 11.13 | | | 6.13 | | | 6.47 | | | 24.59 | |
| | 13(14)-EpDPE | 4.20 | | 0.53 | 2.23 | | 1.05 | 1.88 | | 0.74 | 10.28 | | 5.60 |
| | 13,14-DiHDPE | 0.79 | | 0.08 | 0.73 | | 0.19 | 0.64 | | 0.05 | 0.81 | | 0.09 |
| | Ratio | | 5.30 | | | 3.05 | | | 2.94 | | | 12.66 | |
| | 16(17)-EpDPE | 5.07 | | 0.70 | 2.94 | | 1.31 | 2.44 | | 0.80 | 10.41 | | 5.28 |
| | 16,17-DiHDPE | 2.20 | | 0.35 | 2.28 | | 0.51 | 1.64 | | 0.19 | 2.22 | | 0.20 |
| | Ratio | | 2.30 | | | 1.29 | | | 1.49 | | | 4.68 | |
| | 19(20)-EpDPE | 12.04 | | 2.01 | 8.95 | | 2.32 | 6.61 | | 0.96 | 18.10 | | 6.03 |
| | 19,20-DiHDPE | 6.00 | | 1.14 | 5.50 | | 0.98 | 4.00 | | 0.21 | 6.24 | | 0.58 |
| | Ratio | | 2.01 | | | 1.63 | | | 1.65 | | | 2.90 | |
| total | Epoxyeicosanoid | 497.36 | | | 1014.12 | | | 843.33 | | | 535.16 | | |
| | Di- | 423.15 | | | 322.82 | | | 408.16 | | | 423.59 | | |
| | hydroxyeicosanoid | | | | | | | | | | | | |
| | Ratio | | 1.18 | | | 3.14 | | | 2.07 | | | 1.26 | |

The oxylipins were quantified according to the methods outlined in the text. Drugs were administered s.c., and blood was taken 45 min following administration. Compounds were dissolved in PEG400 and administered at a dose of 1 mg/kg except for pentoxyphilline, which was 10 mg/kg. Below, oxylipins were grouped based on their parent molecules, linoleic acid, ARA, docosahexaenoic acid, and eicosapentaenoic acid (first column). The mean and SE (SEM) of the determined concentration in nM is presented. Ratio of epoxy/dihydroxy eicosanoids for each epoxide/diol pair is shown in red color. The SEM for ratios was omitted for clarity. The graphs presented in FIG. 3 includes sum of ARA, docosahexaenoic acid, and eicosapentaenoic acid metabolites. Table 3 shows for vehicle administered control group.

TABLE 5
Structures and potencies of inhibitors used on recombinant sEH.

| | | Melting | IC50 nM | | | Mass |
|---|---|---|---|---|---|---|
| Name | Structure | point, ° C. | Human | Mouse | Rat | (Da) |
| AUDA | 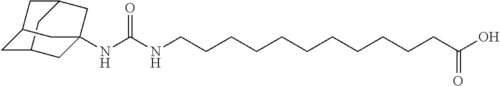 [SMILES: O=C(O)CCCCCCCCCCCNC(=O)NC12CC3CC(CC(C3)C1)C2] | 143 | 3 | 10 | 11 | 392.5 |
| TPAU | 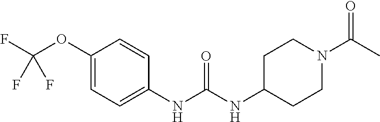 [SMILES: CC(=O)N1CCC(NC(=O)Nc2ccc(OC(F)(F)F)cc2)CC1] | 158 | 12 | 97 | 79 | 345.3 |
| TUPS | 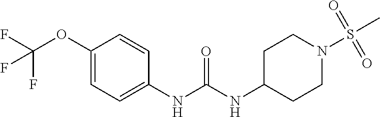 [SMILES: CS(=O)(=O)N1CCC(NC(=O)Nc2ccc(OC(F)(F)F)cc2)CC1] | 237 | 3 | 5 | 9 | 381.4 |
| Rolipram | 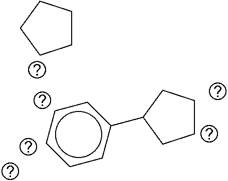 | 132 | >10000 | >10000 | >10000 | 275.3 |

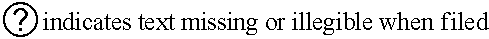

### Example 3

### Pharmacological Characterization of Rolipram and sEHi+Rolipram

Few non-channel, non-neurotransmitter molecules are known to influence sensory function (W. D. Willis, Jr and Coggeshall, R. E., Sensory mechanisms of the spinal cord (Kluwer Academic/Plenum Publishers, New York, 2004), pp. 560)). Therefore it was surprising to find that inhibition of sEH can have a profound effect on nociceptive thresholds (FIG. 3). In order to understand the mechanism of this observation, the pharmacological profile of the interaction between elevated cAMP and epoxy-fatty acids was investigated. To this end, it was investigated whether the effects of the sEHi+rolipram treatment are distinguishable from rolipram alone by using a group of antagonists selected based on our previous work with sEHi (B. Inceoglu et al., Proc Natl Acad Sci USA. 105, 18901 (2008)). First, it was tested if a cox-2 selective inhibitor celecoxib interacted with cAMP elevated by PDEi. Celecoxib (20 mg/kg) at a single dose was administered 30 min prior to increasing doses of rolipram and thermal withdrawal latency was monitored over 4 hr (FIG. 8A). One hour following rolipram administration celebrex did not change rolipram's ability to elevate acute pain behavior indicating that there is minimal interaction between the cyclooxygenase and cAMP pathways. This finding is also consistent with the conclusion that sEHi are distinct pharmacological agents that act through mechanisms independent from suppression of the cyclooxygenase expression (B. Inceoglu et al., Proc Natl Acad Sci USA. 105, 18901 (2008)). Indeed reducing pain produced by PGE₂ also strongly demonstrates that sEHi are a new class of pain reducing agents (FIG. 1A).

Next, based on previous observations that blocking of the steroid synthesis pathway with aminoglutethimide and finasteride were antagonistic to sEHi mediated analgesia it was investigated whether neurosteroids are involved in the mode of action of sEHi (Inceoglu et al., Proc Natl Acad Sci USA. 105, 18901 (2008)). The molecular targets of neurosteroids are believed to be the GABA complex channels (A. L. Morrow, Pharmacology & Therapeutics 116, 1 (2007); A. L. Morrow, in Pharmacology and Therapeutics A. L. Morrow, Ed. (2007), vol. 116 pp. 1-172; D. Belelli, Lambert J J., Nature Reviews Neuroscience 6, 565 (2005)). Accordingly, a GABA_{A} antagonist, picrotoxin was used to test if sEHi augment GABA mediated signaling. A dose of picrotoxin that was inactive by itself was selected to antagonize rolipram and sEHi+ rolipram. This dose of picrotoxin (0.25 mg/kg s.c.) used was not only ineffective on its own in changing pain related behavior but was also possibly too low to cause analgesia by way of inhibiting spinal nociceptive neurons that regulate the descending antinociceptive system (Koyama, et al., Pain 76, 327 (1998)). A third, structurally different sEHi, AUDA, was used in these studies. AUDA, similar to TPAU, was synergistically analgesic when co-administered with rolipram, elevating both thermal withdrawal latency and mechanical withdrawal thresholds, two important measures of pain status (FIGS. 11B and 11C), even though AUDA was ineffective on its own in changing pain related behavior in rats (Inceoglu et al., Life Sciences 79 2311 (2006)). Picrotoxin strongly antagonized the effects of AUDA+rolipram but partially antagonized rolipram (FIG. 11B). Furthermore the effects of picrotoxion were different in regard to antagonizing thermal versus mechanical withdrawal responses (FIG. 11C). This selective antagonism argues that picrotoxin did not act as a stimulant that restored the PDEi suppressed general nervous system activity. Therefore, the involvement of GABA_{A} receptors in sEHi mediated antinociception is proposed. In addition, a neurosteroid synthesis inhibitor and a formerly demonstrated sEH₁ antagonist in an inflammatory pain model, finasteride, acted as a competitive antagonist of rolipram (FIG. 11D).

To further understand the contribution of epoxygenated fatty acids to the analgesic effect of rolipram the de novo synthesis of epoxy-fatty acids was blocked using a CNS permeable cytochrome P450 epoxygenase inhibitor, fluconazole (FIG. 11E). Antagonism produced by fluconazole was non-competitive and non-surmountable. This suggests that only a fraction of the analgesia produced by rolipram is dependent on epoxy-fatty acids. The CNS impermeable epoxygenase inhibitor micanozole completely lacked antagonistic effect strongly arguing that CNS effects of rolipram prevail over peripheral effects. Furthermore, the sEHi treatment in non-inflamed animals led to elevated epoxide/diol ratio but unlike rolipram, not to increases in nociceptive thresholds or motor depression (FIG. 1). Additionally, co-administration of sEHi+rolipram produced an additive increase in the plasma epoxy/diol fatty acid ratio while synergistically elevating nociceptive thresholds. Overall, these observations strongly argue that rolipram acts distinctly from sEHi+rolipram. However, a considerable fraction of rolipram's antinoceiceptive effect seems to be dependent on epoxy-fatty acids.

### REFERENCES

A. A. Spector, A. W. Norris, American Journal of Physiology-Cell Physiology, 00402.2006 (Sep. 20, 2006, 2006).
J. D. Imig, X. Zhao, J. H. Capdevila, C. Morisseau, B. D. Hammock, Hypertension 39, 690 (Feb. 1, 2002, 2002).
B. Inceoglu et al., Life Sciences 79 2311 (2006).
K. R. Schmelzer et al., Proc Natl Acad Sci USA. September 12; 0: 103, 13646 (2006).
B. Inceoglu, K. R. Schmelzer, C. Morisseau, S. L. Jinks, B. D. Hammock, Prostaglandins & Other Lipid Mediators 82, 42 (2007).
M. Terashvili et al., J Pharmacol Exp Ther 326, 614 (Aug. 1, 2008, 2008).
C. Morisseau, B. D. Hammock, Annual Review of Pharmacology and Toxicology 45, 311 (2005).
P. Sura, R. Sura, A. E. EnayetAllah, D. F. Grant, J. Histochem. Cytochem. 56, 551 (Jun. 1, 2008, 2008).
S. G. Khasar, T. Ho, P. G. Green, J. D. Levine, Neuroscience 62, 345 (1994).
P. F. Vonvoigtlander, E. G. Losey, Brain Research 128, 275 (1977).
S. Burstein, G. Gagnon, S. A. Hunter, D. V. Maudsley, Prostaglandins 13, 41 (1977).
U. Zor, Toshio Kaneko, Herman P. G. Schneider, Samuel M. McCann, Irene P. Lowe, Gail Bloom, Barbara Borland, and James B. Field, Proc Natl Acad Sci USA. 63, 918 (1969).
X.-J. Song, Z.-B. Wang, Q. Gan, E. T. Walters, Journal of Physiology 95, 479 (Jan. 1, 2006, 2006).
H. Wachtel, Psychopharmacology 77, 309 (1982).
Y. O. Taiwo, L. K. Bjerknes, E. J. Goetzl, J. D. Levine, Neuroscience 32, 577 (1989).
Y. O. Taiwo, J. D. Levine, Neuroscience 44, 131 (1991).
S. K. Field, Expert Opinion on Investigational Drugs 17, 811 (2008).
L. M. a. Lichtenstein, S. Margolis, Science 161, 902 (Aug. 30, 1968, 1968).
S. Dolan, A. M. Nolan, Neuroscience Letters 309, 157 (2001).
J. A. Siuciak, D. S. Chapin, S. A. McCarthy, A. N. Martin, Psychopharmacology 192, 415 (2007).
J. N. Francischi, C. M. Yokoro, S. Poole, W. L. Tafuri, F. Q., a. M. M. T. Cunha, Eur. J. Pharmacol. 399, 243 (2000).
M. Aoki et al., J Pharmacol Exp Ther 298, 1142 (Sep. 1, 2001, 2001).
D. M. F. Cooper, A. J. Crossthwaite, Trends in Pharmacological Sciences 27, 426 (2006).
W. Krause, Kane, G., Xenobiotica 18, 561 (1988).
K. Node et al., Science 285, 1276 (Aug. 20, 1999).
B. Inceoglu et al., Proc Natl Acad Sci USA. 105, 18901 (2008).
J. R. Vane, R. M. Botting, Inflammation Research 47, 78 (1998).
J. N. Francischi et al., British Journal of Pharmacology 137, 837 (2002).
P. D. Jones, H.-J. Tsai, Z. N. Do, C. Morisseau, B. D. Hammock, Bioorganic & Medicinal Chemistry Letters 16, 5212 (2006).
C. Morisseau, Goodrow, M. H., Newman, J. W., Wheelock, C. E., Dowdy, D. L., Hammock, B. D., Biochemical Pharmacology 63, 1599 (2002).
B. Inceoglu et al., Life Sciences 79 2311 (2006).
B. Inceoglu et al., Proc Natl Acad Sci USA. 105, 18901 (2008).
K. Hargreaves, Dubner, R., Brown, F., Flores, C., Joris, J., Pain 32, 77 (1988).
S. G. Khasar, T. Ho, P. G. Green, J. D. Levine, Neuroscience 62, 345 (1994).
J. Yang, K. Schmelzer, K. Georgi, B. D. Hammock, Analytical Chemistry 81, 8085 (08/28/, 2009).
R. N. Wixtrom, B. D. Hammock, Analytical Biochemistry 174, 291 (1988).
P. D. Jones et al., Analytical Biochemistry 343, 66 (2005).
C. Morisseau, B. D. Hammock, in Techniques for analysis of chemical biotransormation. Current Protocols in Toxicology J. S. Bus, Costa, L. G., Hodgson, E., Lawrence, D. A. and Reed, D. J., Ed. (John Wiley & Sons, New Jersey, 2007) pp. 4.23.1 18.
W. D. Willis, Jr and Coggeshall, R. E., Sensory mechanisms of the spinal cord (Kluwer Academic/Plenum Publishers, New York, 2004), pp. 560.
A. L. Morrow, Pharmacology & Therapeutics 116, 1 (2007).
A. L. Morrow, in Pharmacology and Therapeutics A. L. Morrow, Ed. (2007), vol. 116 pp. 1-172
D. Belelli, Lambert J J., Nature Reviews Neuroscience 6, 565 (2005).
N. Koyama, F. Hanai, T. Yokota, Pain 76, 327 (1998).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of increasing levels of epoxygenated fatty acids in a subject in need thereof comprising administering to the subject an inhibitor of a phosphodiesterase.

2. The method of claim 1, wherein the ratio of epoxygenated fatty acids to dihydroxy fatty acids is increased without changing the levels of dihydroxy fatty acids.

3. The method of claim 1, wherein soluble epoxide hydrolase is not inhibited.

4. The method of claim 1, wherein the inhibitor of phosphodiesterase is an inhibitor of PDE4.

5. The method of claim 4, wherein the inhibitor of PDE4 is selected from the group consisting of rolipram, roflumilast, cilomilast, ariflo, HT0712, ibudilast, mesembrine, pentoxifylline, piclamilast, and combinations thereof.

6. The method of claim 1, wherein the inhibitor of phosphodiesterase is an inhibitor of PDE5.

7. The method of claim 1, wherein the inhibitor of phosphodiesterase is administered in a subtherapeutic dose.

8. The method of claim 1, further comprising administration of an inhibitor of soluble epoxide hydrolase.

9. The method of claim 8, wherein the inhibitor of soluble epoxide hydrolase is administered in a subtherapeutic dose.

10. The method of claim 1, wherein the epoxygenated fatty acids are cis-epoxyeicosantrienoic acids (“EETs”), epoxides of linoleic acid, epoxides of eicosapentaenoic acid (“EPA”) or epoxides of docosahexaenoic acid (“DHA”), or a mixture thereof.

11. A method of obtaining analgesic, anti-convulsant, anti-depressant, anti-inflammatory, anti-hypertensive, cardioprotective, organ protective effects in a subject in need thereof, comprising administering to the subject an inhibitor of phosphodiesterase.

12. A method of reducing, inhibiting, delaying, mitigating, or preventing in a subject pain, seizures, depression, inflammation, hypertension, diabetes, diabetic neuropathy, hyperglycemia, cardiomyopathy, cardiac arrhythmia, cardiac hypertrophy, nephropathy, damage from stroke, chronic obstructive lung diseases, niacin-induced flushing, eye disorders due to increased intraocular pressure and vascular restenosis after angioplasty or stenosis of vascular stents comprising administering to the subject an inhibitor of phosphodiesterase.

13. The method of claim 11, wherein the inhibitor of phosphodiesterase is an inhibitor of PDE4.

14. The method of claim 13, wherein the inhibitor of PDE4 is selected from the group consisting of rolipram, roflumilast, cilomilast, ariflo, HT0712, ibudilast, mesembrine, pentoxifylline, piclamilast, and combinations thereof.

15. The method of claim 11, wherein the inhibitor of phosphodiesterase is an inhibitor of PDE5.

16. The method of claim 11, wherein the inhibitor of phosphodiesterase is administered in a subtherapeutic dose.

17. The method of claim 11, further comprising administration of an inhibitor of soluble epoxide hydrolase.

18. The method of claim 17, wherein the inhibitor of soluble epoxide hydrolase is administered in a subtherapeutic dose.

19. The method claim 11, wherein the epoxygenated fatty acids are cis-epoxyeicosantrienoic acids (“EETs”), epoxides of linoleic acid, epoxides of eicosapentaenoic acid (“EPA”) or epoxides of docosahexaenoic acid (“DHA”), or a mixture thereof.
